(19) United States
(12) Patent Application Publication
(10) Pub. No.: US 2006/0292559 A1
(43) Pub. Date: Dec. 28, 2006

(54) Cell-based microarrays, and methods for their preparation and use

(75) Inventors: M. Parameswara Reddy, Brea, CA (US); Kurt Brillhart, Mission Viejo, CA (US); Daniel Keys, Irvine, CA (US)

Correspondence Address: EDELL, SHAPIRO & FINNAN, LLC, 1901 RESEARCH BLVD., SUITE 400, ROCKVILLE, MD, 20850-3164, US

(73) Assignee: Beckman Coulter, Inc., Fullerton, CA (US)

(21) Appl. No.: 11/448,003

(22) Filed: Jun. 7, 2006

Related U.S. Application Data
(60) Provisional application No. 60/716,486, filed on Sep. 14, 2005.
(60) Provisional application No. 60/693,046, filed on Jun. 23, 2005.

(51) Int. Cl. C12Q 1/70; C12Q 1/68; C12M 1/34

(52) U.S. Cl. 435/5; 435/6; 435/287.2; 977/802; 977/924

(57) ABSTRACT

The present invention is in the field of chemistry and biotechnology. The present invention relates to cell-based microarrays, improved methods for forming such arrays, and methods for using such arrays in diagnostics, therapeutics and research. The invention particularly concerns microarrays in which ligands of a target cells are immobilized to the array support via ligand-binding molecules bound to an oligonucleotide that is hybridized to a support-immobilized oligonucleotide.

### CROSS-REFERENCE TO RELATED APPLICATIONS

This application claims priority to U.S. Provisional Patent Applications Ser. Nos. 60/693,046 (filed on Jun. 23, 2005) and 60/716,486 (filed Sep. 14, 2005), both of which applications are herein incorporated by reference in their entirety.

### FIELD OF THE INVENTION

The present invention is in the field of chemistry and biotechnology. The present invention relates to cell-based microarrays, improved methods for forming such arrays, and methods for using such arrays in diagnostics, therapeutics and research. The invention particularly concerns microarrays in which ligands of target cells are immobilized to the array support via ligand-binding molecules bound to an oligonucleotide that is hybridized to a support-immobilized oligonucleotide.

### BACKGROUND OF THE INVENTION

Assays directed to the detection and quantification of physiologically significant materials in biological fluid and tissue samples are important tools in scientific research and in the health care field.

I. Assays and Microarrays

Several different types of assay have been developed that are capable of detecting relatively high concentrations of components of common biological samples such as human serum (Zhang, T. H., et al., “Detection For Anti-Hantavirus IgM In Patient Serum With Silver Enhanced Dot Immunogold Filtration Assay,” Zhonghua Shi Yan He Lin Chuang Bing Du Xue Za Zhi September 2000;14(3):266-7). Such assays include high-resolution gel electrophoresis (see. e.g., U.S. Patent Appln. Publn. No. US2004/0081979 (Knezevic, V. et al.)) and test procedures based on the catalytic activity of endogeneous enzymes (Bhattacharyya, S. P. et al., “Structural Analysis Of DNA Cleaved In Vivo By Bacteriophage T4 Terminase,” Gene Aug. 19, 1994;146(l):67-72; Gaillot, O. et al., “Molecular Characterization And Expression Analysis Of The Superoxide Dismutase Gene From Streptococcus Agalactiae,” Gene Dec. 19, 1997;204(1-2):213-8; Trigueros, S., et al., “Novel Display Of Knotted DNA Molecules By Two-Dimensional Gel Electrophoresis,” Nucleic Acids Res. Jul. 1, 2001;29(13):E67-7). These methods generally do not have the sensitivity required to detect and quantify the numerous other physiologically important sample constituents which may be present at very low concentrations (e.g., endogeneous molecules intimately involved in cellular regulation (hormones, steroids, biochemical messengers); basic structural components of the organism (amino acids, proteins, polysaccharides); genetic material (DNA, RNA); vitamins, drugs and drug metabolites; toxins, pathogens and substances generated by the immune system). In particular, such methods are generally ill-suited to characterizing or assaying cell surface proteins, such as receptors for hormones, cytokines, immunomodulators (e.g., integrins, selectins, etc.), enzymes, or other molecules.

Microarrays have been widely used in the pharmaceutical and biotechnology industries to permit the simultaneous and coordinated assay of large numbers of analytes (U.S. Patent Appln. Publn. No. US2004/0067539 (Carlsson, R. et al.); Chen, G. Y. et al., “Array-Based Technologies And Their Applications In Proteomics,” Curr. Top. Med. Chem. 2003;3(6):705-724; PCT Publn. No. WO01/69247 (Carlsson, R. et al.); Yeo, D. S. et al., “Strategies For Immobilization Of Biomolecules In A Microarray,” Comb. Chem. High Throughput Screen. 2004 May;7(3):213-221). Such assays are particularly useful in characterizing gene and protein expression patterns in human disease processes in order to identify candidate therapeutic agents.

Oligonucleotide microarrays typically involve the micropatterned deposition of oligonucleotides and detect the hybridization of complementary oligonucleotides (see, e.g., Chittur, S. V. “DNA Microarrays: Tools For The 21st Century,” Comb. Chem. High Throughput Screen. Sep. 2004;7(6):531-537; Sarang, S. S. et al., “Discovery Of Molecular Mechanisms Of Neuroprotection Using Cell-Based Bioassays And Oligonucleotide Arrays,” Physiol. Genomics Oct. 29, 2002;11(2):45-52; Epstein, J. R. et al., “High-Density, Microsphere-Based Fiber Optic DNA Microarrays,” Biosens Bioelectron. May 2003;18(5-6):541-546; Chen, G. Y. et al., “Array-Based Technologies And Their Applications In Proteomics,” Curr. Top. Med. Chem. 2003;3(6):705-724; Wells, J. M. “Genes Expressed In The Developing Endocrine Pancreas And Their Importance For Stem Cell And Diabetes Research,” Diabetes Metab. Res. Rev. May-June 2003;19(3):191-201; Reilly, S. C. et al., “Discovering Genes: The Use Of Microarrays And Laser Capture Microdissection In Pain Research,” Brain Res. Brain Res. Rev. October 2004;46(2):225-233; Khetani, S. R. et al., “Exploring Interactions Between Rat Hepatocytes And Nonparenchymal Cells Using Gene Expression Profiling,” Hepatology. September 2004;40(3):545-554; Hardiman, G., “Microarray Platforms-Comparisons And Contrasts,” Pharmacogenomics July 2004;5(5):487-502; Meloni, R. et al., “DNA Microarrays And Pharmacogenomics,” Pharmacol. Res. April 2004;49(4):303-308; Kultima, K. et al., “Valproic Acid Teratogenicity: A Toxicogenomics Approach,” Environ. Health Perspect. August 2004;112(12):1225-1235).

Antibody and protein microarrays have also been described as an alternative to low throughput protein interaction studies, such as ELISA, for conducting the global analysis of the protein complement of a target cell (Panicker, R. C. et al., “Recent Advances In Peptide-Based Microarray Technologies,” Comb. Chem. High Throughput Screen. September 2004;7(6):547-556; Pavlickova, P. et al., “Advances In Recombinant Antibody Microarrays,” Clin. Chim. Acta. May 2004;343(1-2):17-35); Chen, G. Y. et al., “Array-Based Technologies And Their Applications In Proteomics,” Curr. Top. Med. Chem. 2003;3(6):705-724; Nielsen, U. B. et al., “Multiplexed Sandwich Assays In Microarray Format,” J. Immunol. Methods July 2004;290(1-2):107-120; Bailey, S. N. et al., “Microarrays Of Small Molecules Embedded In Biodegradable Polymers For Use In Mammalian Cell-Based Screens,” Proc. Natl. Acad. Sci. U.S.A. Nov. 16, 2004;101(46):16144-9. Epub Nov. 16, 2004; U.S. Patent Applns. Publn. Nos. US2004/0033546 (Wang, D.), US2003/0153013 (Huang, R. P.); US2003/0108972 (Zweig, S. E. et al.); US2003/0108949 (Bao, G. et al.); 2002/0164656 (Hoeffler, J. P. et al.); PCT Publn. WO99/40434 (Hoeffler, J. P. et al.); PCT Publn. No. WO2004/076678 (Green, L.); PCT Publn. No. WO2004/005477 (Charych, D. et al.); PCT Publn. No. WO02/073180 (Huang, R. P.); PCT Publn. No. WO02/39120 (George, S. T. et al.); PCT Publn. No. WO02/12893 (Cardone, M. H. et al.); PCT Publn. No. WO00/63701 (Brown, P. et al.); PCT Publn. No. WO03/003014 (Pearce, C. D. J. et al.)). Hydrogel-based microarrays are disclosed in PCT Publn. No. WO02/083918 (Wang, D.)). Phage-based microarrays are discussed in PCT Publn. No. WO01/36585 (Anderson, N. L.). The capacity of complementary oligonucleotides to anneal to one another has led to the use of oligonucleotide tagged proteins as a means for converting an oligonucleotide microarray into a protein array (see. e.g., Reddy, M. P. et al., U.S. Pat. No. 5,648,213; Jackson, A. M. et al., “Cell-Free Protein Synthesis For Proteomics,” Brief Funct. Genomic Proteomic February 2004;2(4):308-319); Oleinikov, A. V. et al., “Self-Assembling Protein Arrays Using Electronic Semiconductor Microchips And In Vitro Translation,” J. Proteome Res. May-June 2003;2(3):313-319; Weng, S. et al., “Generating Addressable Protein Microarrays With Profusion Covalent mRNA-Protein Fusion Technology,” Proteomics January 2002;2(1):48-57).

Cell-based microarrays pen-nit an investigation of the impact of conditions or target reagents on living cells, and are increasingly being used in pharmaceutical studies as an intermediate step between inexpensive receptor-based assays and expensive tissue and animal based studies. The cells of such microarrays are immobilized to the microarray support by covalently bonding cell-binding antibodies to the support (Ko, K. et al., “Antibody Microarray For Correlating Cell Phenotype With Surface Marker” Biomaterials 26(6)687-696, 2005-(e-pub 2004)), by printing small aliquots of cells to the solid support (Delehanty, J. B. et al., “A Comparison Of Microscope Slide Substrates For Use In Transfected Cell Microarrays,” Biosens Bioelectron. Nov. 1, 2004;20(4):773-779; Ziauddin, J. et al., “Microarrays Of Cells Expressing Defined cDNAs,” Nature May 3, 2001;411(6833):107-110), by crosslinking or other coating reagents (Chen, G. Y. et al., “Array-Based Technologies And Their Applications In Proteomics,” Curr. Top. Med. Chem. 2003;3(6):705-724); Otsuka, H. et al., “Two-Dimensional Multiarray Formation Of Hepatocyte Spheroids On A Microfabricated PEG-Brush Surface,” Chembiochem. Jun. 7, 2004;5(6):850-855; Honma, K. et al., “Atelocollagen-Based Gene Transfer In Cells Allows High-Throughput Screening Of Gene Functions,” Biochem. Biophys. Res. Commun. Dec. 21, 2001;289(5):1075-1081; Kato, K. et al., “Transfection Microarray Of Nonadherent Cells On An Oleyl Poly(Ethylene Glycol) Ether-Modified Glass Slide,” Biotechniques September 2004;37(3):444-8, 450, 452), or through physical means such as “cratering” the support (Xu, C. W. “High-Density Cell Microarrays For Parallel Functional Determinations,” Genome Res. March 2002;12(3):482-486). Methods have been described for making uniform micro-patterned arrays of cells for other applications, for example photochemical resist-photolithograpy. (Mrksich, M. et al., “Using Self-Assembled Monolayers To Understand The Interactions Of Man-Made Surfaces With Proteins And Cells,” Annu Rev Biophys Biomol Struct. 1996;25:55-78). Reactive ion etching has been similarly used on the surface of silicon wafers to produce surfaces patterned with two different types of texture (Craighead, H. G. et al., “Textured Thin-Film Si Solar Selective Absorbers Using Reactive Ion Etching,” Appl. Phys. Lett. 37:653, 1980; Craighead, H. G. et al., “Textured Surfaces—Optical Storage and Other Applications,” J. Vac. Sci. Technol. 20:316, 1982; Suh, s. Y. et al., “Morphology Dependent Contrast Measurements Of Microscopically Textured Germanium Films,” Proc. SPIE 382:199, 1983). Photoresist stamping has been used to produce cell-based microarrays (Singhvi R. et al., “Engineering Cell Shape And Function,” Science 264:696-698, 1994). An elaboration involving strong, but non-covalent, metal chelation has been used to coat gold surfaces with patterns of specific proteins (Sigal, G. B. et al. “A Self-Assembled Monolayer For The Binding And Study Of Histidine-Tagged Proteins By Surface Plasmon Resonance,” Anal. Chem. 68:490-497, 1996). U.S. Pat. No. 6,103,479 (Taylor, D. L.) and PCT Publn. No. WO03/102578 (Van Damme, H. et al.) disclose methods of forming and using high throughput cell-based microarrays. U.S. Patent Appln. Publn. No. US20030157523 (Franz, G.) discloses arrays of cells produced by depositing frozen materials within individual wells of sectionable material, resulting in stainable sections of non-living cells.

Tissue-based microarrays have also been described (see, e.g., Braunschweig, T. et al., “Perspectives In Tissue Microarrays,” Comb. Chem High Throughput Screen. September 2004;7(6):575-585; Shergill, I. S. et al., “Tissue Microarrays. A Current Medical Research Tool,” Curr. Med. Res. Opin. May 2004;20(5):707-712); PCT Publn. No. WO02/48674 (Knezevic, V. et al.)).

II. Bioconjugates

Bioconjugates, such as protein-oligonucleotide conjugates, are employed in a wide variety of molecular biology applications (see, Reddy, M. P. et al., U.S. Pat. No. 5,648,213; Farooqui, F. et al., U.S. patent application Ser. No. 10/032,592; U.S. Patent Appln. Publn. No. 20050164292). For example, bioconjugates such as oligonucleotides conjugated to antibodies or enzymes have been used as hybridization probes in immunoassays (U.S. Pat. No. 5,648,213 (Reddy, M. P. et al.); Ghosh, S. S. , et al., “Use Of Maleimide-Thiol Coupling Chemistry For Efficient Syntheses Of Oligonucleotide-Enzyme Conjugate Hybridization Probes,” Bioconjug Chem January-February 1990;1(1):71-6; Keller and Manak, DNA Probes, 2nd Edition (Stockton Press, New York, 1993; Milligan et al., “Current Concepts In Antisense Drug Design,” J. Med. Chem., 36: 1923-1937 (1 993); Drmanac et al, Science, 260: 1649-1652 (1993); Bains, J., DNA Sequencing and Mapping, 4: 143-150 (1993)). They have been used in diagnostic assays to improve assay sensitivity (U.S. Pat. No. 6,197,513 (Coull, et al.). Oligonucleotide-antibody conjugates have also been used as probes in the development of sensitive nucleic acid-based diagnostic assays (Martin R., et al., “A Highly Sensitive, Nonradioactive DNA Labeling And Detection System,” 13: Biotechniques December 1990;9(6):762-8) (Podbielski A, et al., “Identification Of Group A Type 1 Streptococcal M Protein Gene By A Non-Radioactive Oligonucleotide Detection Method,” 14: Med. Microbiol. Immunol. (Berl.) 1990;179(5):255-62; Carpenter W. R., et al., “A Transcriptionally Amplified DNA Probe Assay With Ligatable Probes And Immunochemical Detection,” 9: Clin. Chem. September 1993;39(9):1934-8). Other bioconjugates, such as isothiocyanates (ITCs) conjugates, are used in bioassays as versatile chemopreventive agents (Chung E. L., “Chemoprevention Of Lung Cancer By Isothiocyanates And Their Conjugates In A/J Mouse,” Exp Lung Res April-May 2001;27(3):319-30). Protein-polysaccharide conjugates with reciprocally enhanced immunogenicity have been used in the development of combination vaccines (Gupta R. K., et al., “Adjuvants For Human Vaccines—Current Status, Problems And Future Prospects,” Vaccine October 1995;13(14):1263-76).

The preparation of bioconjugates involves multiple steps that require the protein, oligonucleotide, or both, to be modified with the appropriate linking moiety and then purified before being combined and reacted with each other. Such conjugates have traditionally been prepared by methods, such as glutaraldehyde crosslinking, maleimide-thiol coupling (Ghosh, S. S. , et al., “Use Of Maleimide-Thiol Coupling Chemistry For Efficient Syntheses Of Oligonucleotide-Enzyme Conjugate Hybridization Probes,” Bioconjug. Chem. January-February 1990;1(1):71-6), isothiocyanate-amine coupling (Brandtzaeg, “Conjugates Of Immunoglobulin G With Different Fluorochromes. I. Characterization By Anionic-Exchange Chromatography,” Scand. J. Immunol. 2: 273-290 1973; Loken, M. R. et al., “Analysis Of Cell Populations With A Fluorescence-Activated Cell Sorter,” 1975, Annals N.Y. Acad. Sci. 254: 163-171; U.S. Pat. No. 5,648,213 (Reddy, M. P. et al.); Keller, G. H., et al., “DNA Probes,” MacMillan Publishers Ltd., 1989), and Schiff base formation/reduction. Often the modification reaction results in an unstable reactive enzyme or oligomer intermediate that must be purified and used immediately. For these and other reasons, the yield of conjugate is highly variable when these techniques are used. Furthermore, reaction times are lengthy, and several purification steps are generally needed to obtain a purified conjugate. Finally, in most instances a portion of the enzymatic activity is lost due to the nature of the chemical reactions, lengthy reaction times, and numerous purification steps.

Despite all such advances, a need continues to exist for compositions and methods that can be used to achieve the conjugation of target cells to a surface in a manner that reflects the expression and array of the target cell's ligands. More specifically, a need exists for compositions and methods suitable for immobilizing ligands of target cells to ligand-binding molecules that have been bound to a solid support. The present invention is directed to such a need.

### SUMMARY OF THE INVENTION

The present invention is in the field of chemistry and biotechnology. The present invention relates to cell-based microarrays, improved methods for forming such arrays, and methods for using such arrays in diagnostics, therapeutics and research. The invention particularly concerns microarrays in which ligands of target cells are immobilized to the array support via ligand-binding molecules bound to an oligonucleotide that is hybridized to a support-immobilized oligonucleotide.

In detail, the invention provides a cell-based microarray, comprising:
(A) a target cell having a surface ligand;
(B) one or more species of bioconjugate molecules, each such molecules comprising a ligand-binding molecule portion conjugated to an oligonucleotide molecule portion, and each such species having a different ligand-binding portion, and
(C) a planar or non-planar support (e.g., glass, paper, optical fiber, plastic, a bead, etc.) having immobilized thereto one or more species of oligonucleotide molecules, each such species having different a different nucleotide sequence,
   wherein an oligonucleotide portion of a bioconjugate molecule and a support-immobilized oligonucleotide are hybridized to one another, and wherein the ligand-binding molecule of the hybridized bioconjugate molecule is bound to the surface ligand of the target cell, thereby immobilizing the target cell to the support. wherein the oligonucleotide portion of the bioconjugate and the support-immobilized oligonucleotide are hybridized to one another, and wherein the ligand-binding molecule is bound to the surface ligand of the target cell, thereby immobilizing the target cell.

The invention concerns the embodiments of such a cell-based microarray wherein the target cell is a mammalian cell (especially a human cell), a reptilian cell, an avian cell, a fish cell, a fungal cell, a plant cell, a yeast cell, a bacterial cell, or viral particle.

The invention additionally concerns the embodiments of such cell-based microarrays wherein two or more oligonucleotide molecules having differing oligonucleotide sequences are bound to the support and/or wherein the different species of bioconjugate molecules have different ligand-binding molecule portions or different oligonucleotide sequences. The invention additionally concerns the embodiments of such cell-based microarrays wherein the two or more different species of bioconjugate molecules have different ligand-binding molecule portions and/or different oligonucleotide sequences.

The invention additionally concerns the embodiments of such cell-based microarrays wherein the microarray comprises a plurality of different species of target cells each such species bound to a different species of bioconjugate molecule, wherein the different species of bioconjugate molecule are hybridized to an ordered array of oligonucleotides immobilized to the support.

The invention additionally concerns the embodiments of such cell-based microarrays wherein the surface ligand is an antigenic surface protein, a receptor, a transmembranous enzyme, that is naturally present on the surface of normal or on abnormal target cells. The invention additionally concerns the embodiments of such cell-based microarrays wherein the presence of the surface ligand is associated with a disease state or a morphological state (such as an apoptotic state).

The invention additionally concerns the embodiments of such cell-based microarrays wherein the ligand binding molecule is an immunoglobulin, a hormone, an immunomodulator, a cytokine, a chemokine, a pharmacological agent or a substrate or inhibitor of a transmembranous enzyme.

The invention additionally concerns the embodiments of such cell-based microarrays wherein a molecule of a species of one of the bioconjugate molecules is formed by a method that comprises the steps of:

(A) contacting an oligonucleotide having an amino group with a heterofunctional linker, wherein the linker has a first group reactive with the amino group and a second group reactive with a thiol group, the contacting being under conditions sufficient to permit the first group of the heterofunctional linker to become bonded to the amino group of the oligonucleotide, thereby forming an oligonucleotide-heterofunctional linker conjugate; and

(B) contacting the oligonucleotide-heterofunctional linker conjugate (A) with a protein having a thiol group reactive with the second group of the heterofunctional linker; the contacting being under conditions sufficient to permit the thiol group of the protein to become bonded to the second group of the heterofunctional linker of the oligonucleotide-heterofunctional linker conjugate, to thereby form a molecule of a species of the bioconjugate molecules.

The invention additionally concerns the embodiments of such method of forming cell-based microarrays wherein the first group of the heterofunctional linker is an NHS group (especially Sulfo-SMCC; Sulfo-EMCS; Sulfo-GMBS; Sulfo-KMUS; Sulfo-MBS; Sulfo-SIAB; Sulfo-SMPB; Sulfo-LC-SMPT; SVSB; SIACX; SIA, SIAXX; and NPIA), and the second group of the heterofunctional linker is a maleimide group.

The invention additionally concerns the embodiments of such cell-based microarrays wherein the assay assays the viability of the target cell, and/or wherein the assay assays the presence or expression of an internal component of the target cell (especially, a component of a cell signaling pathway, a G-Protein Coupled Receptor or an indicator of inflammation), especially wherein the presence or expression of the internal component is characteristic of a morphologic (e.g., an apoptotic) state or a disease state.

The invention additionally provides a method for determining whether a population of cells contains a target cell that possesses a desired surface ligand, the method comprising the steps:

(A) incubating the population of cells in the presence of:
(1) one or more species of bioconjugate molecules, each such species comprising a ligand-binding molecule portion conjugated to an oligonucleotide molecule portion, wherein at least one of the species of bioconjugate molecules comprises a ligand-binding molecule portion capable of binding to the desired surface ligand; and
(2) a support having immobilized thereto one or more species of oligonucleotide molecules, each such species having a different nucleotide sequence, and at least one species being capable of hybridizing to the nucleotide sequence of the oligonucleotide of the bioconjugate molecule, wherein the incubation is conducted under conditions sufficient to permit:
   (a) hybridization between complementary nucleotide sequences of the bioconjugate and the support-immobilized oligonucleotide; and
   (b) binding between a ligand-binding molecule of the bioconjugate molecules and the desired surface ligand of the target cell to thereby immobilize the target cell to the support; and

(B) determining whether any cell of the population possess the surface ligand by detecting the immobilization of cells to the surface, wherein the immobilization is achieved through the binding of the target cell's surface ligand to the ligand-binding molecule of a bioconjugate whose oligonucleotide portion has hybridized to a support-immobilized oligonucleotide.

The invention additionally concerns the embodiments of such method, wherein two or more oligonucleotide molecules having differing oligonucleotide sequences are bound to the support and/or wherein two or more different species of bioconjugate molecules are bound to the support, and wherein the different species of bioconjugate molecules have different ligand-binding molecule portions or different oligonucleotide sequences. The invention also concerns the embodiments of such methods wherein the two or more different species of bioconjugate molecules have different ligand-binding molecule portions, and/or wherein the two or more different species of bioconjugate molecules have different oligonucleotide sequences.

The invention also provides the embodiments of such methods wherein the microarray comprises a plurality of different species of target cells each such species bound to a different species of bioconjugate molecule, wherein the different species of bioconjugate molecule are hybridized to an ordered array of oligonucleotides immobilized to the support.

The invention also provides the embodiments of such methods wherein the target cell is a mammalian cell (especially a human cell), a reptilian cell, an avian cell, a fish cell, a fungal cell, a plant cell, a yeast cell, a bacterial cell, or a viral particle.

The invention also provides the embodiments of such methods wherein the surface ligand is an antigenic surface protein, a receptor, a transmembranous enzyme, that is naturally present on the surface of normal target cells, wherein the surface ligand is an antigenic surface protein, a receptor, a transmembranous enzyme, that is naturally present on the surface of abnormal target cells; and/or wherein the presence of the surface ligand is associated with a disease state (e.g., cancer).

The invention also provides the embodiments of such methods wherein the target cell is detectably labeled, especially with a detectably labeled ligand-binding molecule or a detectably labeled cell.

The invention also provides the embodiments of such methods wherein the ligand binding molecule is an immunoglobulin, a hormone, an immunomodulator, a cytokine, a chemokine, a pharmacological agent or a substrate or inhibitor of a transmembranous enzyme.

The invention also provides the embodiments of such methods wherein the solid support is an optical waveguide, and wherein the detection of immobilization of target cells to the surface is preformed by measuring a detectable label using a fiber optic waveguide detector.

The invention also provides the embodiments of such methods wherein in step (A), the surface ligand of the target cells is permitted to bind to the ligand-binding molecule portion of the bioconjugate molecule prior to permitting the oligonucleotide portion of the bioconjugate molecule to hybridize to the support-immobilized oligonucleotide; and/or wherein in step (A), the surface ligand of the target cells is permitted to bind to the ligand-binding molecule portion of the bioconjugate molecule and the oligonucleotide portion of the bioconjugate molecule is permitted to hybridize to the support-immobilized oligonucleotide simultaneously; and/or wherein in step (A), the oligonucleotide portion of the bioconjugate molecule is permitted to hybridize to the support-immobilized oligonucleotide prior to permitting the surface ligand of the target cells to bind to the ligand-binding molecule portion of the bioconjugate molecule.

The invention also provides the embodiments of such methods wherein the determination is accomplished by detecting the presence of a detectable label, especially wherein the detectable label is a ligand-binding molecule and/or a detectably labeled cell.

The invention also provides the embodiments of such methods wherein the presence of the detectable label is determined without dissociating the hybridized oligonucleotide molecules, and/or after dissociating the hybridized oligonucleotide molecules.

The invention also provides the embodiments of such methods wherein the hybridized oligonucleotides are dissociated using a cleaving reagent selected from the group consisting of ionized water, a urea-containing solution, a formamide-containing solution, and an oligonucleotide.

The invention also provides the embodiments of such methods wherein a molecule of a species of one of the bioconjugate molecules is formed by a method that comprises the steps of:
(A) contacting an oligonucleotide having an amino group with a heterofunctional linker, wherein the linker has a first group reactive with the amino group and a second group reactive with a thiol group, the contacting being under conditions sufficient to permit the first group of the heterofunctional linker to become bonded to the amino group of the oligonucleotide, thereby forming an oligonucleotide-heterofunctional linker conjugate; and
(B) contacting the oligonucleotide-heterofunctional linker conjugate (A) with a protein having a thiol group reactive with the second group of the heterofunctional linker; the contacting being under conditions sufficient to permit the thiol group of the protein to become bonded to the second group of the heterofunctional linker of the oligonucleotide-heterofunctional linker conjugate, to thereby form a molecule of a species of the bioconjugate molecules.

The invention also provides the embodiments of such methods wherein the first group of the heterofunctional linker is an NHS group (especially Sulfo-SMCC; Sulfo-EMCS; Sulfo-GMBS; Sulfo-KMUS; Sulfo-MBS; Sulfo-SIAB; Sulfo-SMPB; Sulfo-LC-SMPT; SVSB; SIACX; SIA, SIAXX; and NPIA), and the second group of the heterofunctional linker is a maleimide group.

The invention also provides the embodiments of such methods wherein the assay assays the viability of the target cell, and/or the presence or expression of an internal component of the target cell (especially a component of a cell signaling pathway, a G-Protein Coupled Receptor or an indicator of inflammation.

The invention also provides the embodiments of such methods wherein the presence or expression of the internal component is characteristic of an apoptotic state or a disease state.

The invention also provides a method for identifying a ligand-binding molecule that binds to a surface ligand of a cell, the method comprising the steps:
(A) incubating a population of cells that possess the surface ligand in the presence of:
   (1) a candidate ligand-binding molecule suspected of being capable of binding to the surface ligand of the cell;
   (2) a bioconjugate molecule, the bioconjugate molecule comprising a ligand-binding molecule portion conjugated to an oligonucleotide molecule portion; and
   (3) a support having immobilized thereto an oligonucleotide molecule;
   wherein the bioconjugate oligonucleotide and the support-immobilized oligonucleotide are capable of hybridizing to one another, and wherein the incubation is conducted under conditions sufficient to permit:
   (a) the bioconjugate oligonucleotide and the support-immobilized oligonucleotide to hybridize to one another; and
   (b) the ligand-binding molecule portion of the bioconjugate to bind to the surface ligand of the cell; and
(B) determining whether the presence of the candidate ligand-binding molecule affects the extent of immobilization of the cells to the solid support.

The invention also provides the embodiments of such methods wherein in step (A): the surface ligand of the target cells is permitted to bind to the ligand-binding molecule portion of the bioconjugate molecule prior to permitting the oligonucleotide portion of the bioconjugate molecule to hybridize to the support-immobilized oligonucleotide; the surface ligand of the target cells is permitted to bind to the ligand-binding molecule portion of the bioconjugate molecule and the oligonucleotide portion of the bioconjugate molecule is permitted to hybridize to the support-immobilized oligonucleotide simultaneously; and/or the oligonucleotide portion of the bioconjugate molecule is permitted to hybridize to the support-immobilized oligonucleotide prior to permitting the surface ligand of the target cells to bind to the ligand-binding molecule portion of the bioconjugate molecule.

The invention also provides a method for determining whether a population of cells contains a target cell that possesses a desired internal molecule, the method comprising the steps:

(A) incubating the population of cells in the presence of:
(1) a bioconjugate molecule comprising a ligand-binding molecule portion conjugated to an oligonucleotide molecule portion; and
(2) a support having immobilized thereto an oligonucleotide molecule,
wherein the bioconjugate oligonucleotide and the support-immobilized oligonucleotides are capable of hybridizing to one another; and wherein the incubation is conducted under conditions sufficient to permit:
   (a) the bioconjugate oligonucleotide and the support-immobilized oligonucleotide to hybridize to one another; and
   (b) the ligand-binding molecule portion of the bioconjugate to bind to the surface ligand of the target cell to thereby immobilize cells to the support; and

(B) determining whether any immobilized cell of the population possess the desired internal ligand by detecting the presence of the desired ligand within immobilized target cells.

The invention additionally concerns the embodiments of such method wherein the microarray assays for the presence of a nucleic acid molecule produced within the immobilized cell, and/or wherein the assayed nucleic acid molecule is amplified via an in vitro nucleic acid amplification process.

The invention also provides a method for screening for a desired molecule (especially a pharmacological agent or a cosmetic) comprising:
(A) incubating a candidate desired molecule in the presence of a microarray of cells immobilized to a solid support, wherein the microarray is formed by incubating a population of cells that possess a surface ligand in the presence of:
   (1) a ligand-binding molecule capable of binding to the surface ligand of the cell;
   (2) a bioconjugate molecule comprising a ligand-binding molecule portion conjugated to an oligonucleotide molecule portion; and
   (3) a support having immobilized thereto an oligonucleotide molecule;
   wherein the support has immobilized thereon an oligonucleotide molecule that hybridizes to the oligonucleotide portion of the bioconjugate and the ligand-binding molecule portion of the bioconjugate binds to the surface ligand of the cell so as to immobilize the cells to the solid support; and
(B) determining whether the presence of the candidate desired molecule affects the extent of immobilization of the cells to the solid support.

### BRIEF DESCRIPTION OF THE FIGURES

FIG. 1 illustrates the synthesis of an oligonucleotide-antibody conjugate. The preparation begins with the synthesis of the 3′-amino oligonucleotide that is activated with a hetero-bifunctional linker sulfo SMCC. Antibodies are then thiolated using Traut's reagent (iminothiolane). The activated oligonucleotide and thiolated antibodies are subsequently mixed to facilitate coupling of oligonucleotide to antibody.

FIG. 2 illustrates a direct cell-based microarray of the present invention in which a molecule that interacts with a cell surface marker, in this case an antibody (2) is attached to an oligonucleotide (1). The resulting bioconjugate (3) is incubated with an oligonucleotide microarray (4) and hybridizes to the specific site containing the complementary sequence (5) to form a microarray (6). Use of multiple conjugates produces a microarray of various molecular species. When cells (7, 8) are added to the microarray, cells with the specific surface marker (7) recognized by antibody (2) are immobilized at the corresponding site on the microarray. Other cells are not immobilized.

FIG. 3 illustrates an indirect cell-based microarray of the present invention in which a molecule that interacts with a cell surface marker, in this case an antibody (22) capable of binding to a surface molecule of cell (27), is attached to an oligonucleotide (21) to form bioconjugate (23). Bioconjugate (23) is incubated with oligonucleotide array (24) having specific oligonucleotide species (25) capable of hybridizing to oligonucleotide (21) thereby forming microarray (26). Cells (27) and (28) are incubated in the presence of microarray (26). Labeled antibody (29) binds to microarray-immobilized cell (27), thereby permitting its detection and quantification.

FIG. 4A and FIG. 4B illustrate the use of oligonucleotide linker technology to generate microarrays (46, 47). Microarray (46) contains sites with an immobilized drug (40) (immobilized for example via an oligonucleotide-drug conjugate) that interacts with cell surface receptor (41) of cell (39). Microarray (47) contains antibodies (42) to a soluble protein (43) produced by cells in the sample. Following incubation with the sample and labeled antibodies (44, 45) to cell surface receptor (41) and soluble protein (43), both species can be independently or concurrently detected and/or quantified. The sandwich microarrays shown in FIGS. 4A and 4B may comprise arrays in communication with one another, or regions or zones of the same microarray. The microarrays shown in FIGS. 4A and 4B alternatively depict two embodiments of the sandwich cell-based microarrays of the present invention.

FIG. 5 illustrates the use of a detectably labeled reagent to detect the immobilization of target cells in a microarray of the present invention. Oligonucleotide (51) is attached to antibody (52). The resulting bioconjugate (53) is incubated with an oligonucleotide microarray (54) and hybridizes to the specific site containing the complementary sequence (55) to form a microarray having immobilized antibody (56). Use of multiple conjugates produces a microarray of various molecular species. When cells (57, 58, 59) are added to the microarray, cells (57, 58) having the specific surface marker recognized by antibody (52) are immobilized at the corresponding site on the microarray. Other cells (59) are not immobilized. Use of different detectably labeled reagents specific to distinct cell surface molecules of the immobilized cells can be used to detect, differentiate or quantitate the sub-populations of immobilized cells.

FIG. 6 illustrates the use of a cell (78) having the ability to bind to the target cell (77) to detect the immobilization of target cells in a microarray of the present invention. Oligonucleotide (71) is attached to antibody (72). The resulting bioconjugate (73) is incubated with an oligonucleotide microarray (74) and hybridizes to the specific site containing the complementary sequence (75) to form a microarray having immobilized antibody (76). Use of multiple conjugates produces a microarray of various molecular species. When cells (77, 78) are added to the microarray, cells (77) having the specific surface marker recognized by antibody (72) are immobilized at the corresponding site on the microarray. Detection of binding is achieved using cells (78) that are capable of binding to the immobilized target cell. Detection of immobilized cells (78) is thus indicative of the binding of target cells (77).

FIG. 7 illustrates an immunochromatographic microarray format capable of assaying for agents capable of disrupting the binding of a target cell to a microarray support having two separated but connected or adjacent microarray regions (86, 88). Target cell (81) possessing surface ligand molecules (82, 83, 84) is incubated in the presence of microarray region (86) to which an excess of ligand-binding molecule (85) has been immobilized. Binding between ligand-binding molecule (85) and cell ligand (83) immobilizes target cell (81) to microarray (86). Candidate agent (89) is introduced. Candidate agent (89) competes with cell ligand (83) for binding to immobilized ligand-binding molecule (85), disrupting the target cell immobilization. Detectably labeled antibody (90) binds to ligand (82). Microarray region (86) is then incubated under conditions (e.g., washing, fluid flow, etc.) sufficient to permit non-immobilized cells to separate from immobilized cells and encounter microarray region (88) (hence the chromatographic aspect of the immunoassay). When non-immobilized cells come into contact with microarray (88), target cells possessing ligand (84) become immobilized to microarray (88) through binding of their ligand (84) to immobilized antibody (87) of microarray (88). Detection of immobilized cell in microarray region (88) indicates that candidate agent (89) was capable of disrupting the binding between ligand (83) and ligand-binding molecule (85).

FIG. 8 illustrates an immunochromatographic microarray format capable of assaying for target cells capable of disrupting the binding of detectably labeled molecule (e.g., a candidate pharmacological agent, a hormone, a soluble receptor ligand, etc.) to a microarray support having two separated but connected or adjacent microarray regions (98, 99). Molecule (94), having detectably label (96) is incubated in the presence of microarray region (98) to which an excess of ligand-binding molecule (95) has been immobilized. Detectable label (96) may be a direct label (e.g., a radioactive, fluorescent, enzymatic, etc. label) or an indirect label (e.g., an antibody, etc.). Binding between ligand-binding molecule (95) and molecule (94) immobilizes molecule (94) to microarray (98). Target cell (91) possessing surface ligands (92, 93) is introduced. Target cell (91) competes with molecule (94) for binding to immobilized ligand-binding molecule (95), disrupting molecule (94) immobilization. Microarray region (98) is then incubated under conditions (e.g., washing, fluid flow, etc.) sufficient to permit non-immobilized molecule (94) to separate from immobilized cells and encounter microarray region (99) (hence the chromatographic aspect of the immunoassay). When non-immobilized molecules come into contact with microarray region (99), molecule (94) binds to antibody (97) and becomes immobilized to microarray region (99). Detection of immobilized molecule (94) in microarray region (99) indicates that target cell (91) was capable of disrupting the binding between molecule (94) and ligand-binding molecule (95).

### DESCRIPTION OF THE PREFERRED EMBODIMENTS

The present invention is in the field of chemistry and biotechnology. The present invention relates to cell-based microarrays, improved methods for forming such arrays, and methods for using such arrays in diagnostics, therapeutics and research. The invention particularly concerns microarrays in which ligands of a target cells are immobilized to the array support via ligand-binding molecules bound to an oligonucleotide that is hybridized to a support-immobilized oligonucleotide.

Recently cell-based arrays have been utilized by the pharmaceutical industry for drug screening. These serve as a relatively inexpensive intermediate step between receptor-based studies (such as immunoassays) and expensive tissue and organism studies. Ko et al., have demonstrated that cell-based arrays can be produced by incubating living cells with microarrays of antibodies to cell-surface markers (Ko, K. et al., “Antibody Microarray For Correlating Cell Phenotype With Surface Marker” Biomaterials 26(6)687-696, 2005 (e-pub 2004)). The study used microarrays produced by linking antibodies directly to the surface.

As described in greater detail below, the microarrays of the present invention can be used to identify or fractionate (or otherwise concentrate or purify) desired target cells. They may be used to detect the presence or absence of an abnormality (especially a morphological state (such as an apoptotic state) or a disease state (such as a tumorigenic state)) as evidenced by cell surface molecules, or alternatively, by assaying the activity of internal enzymes. The microarrays of the present invention may be used to assay cell type (e.g., CD4+ lymphocytes, etc.). They may also be used in assays for pharmacological agents, hormones and other biological molecules.

### I. PREFERRED CHARACTERISTICS OF THE MICROARRAYS OF THE INVENTION

The microarrays of the present invention are preferably formed by immobilizing a target cell to a solid support. The target cells may be viable, non-viable, permeabilized, quiescent, metabolically active, induced, repressed, etc. depending upon the desire of the user. Where viable cells are desired, the microarray may be incubated in the presence of suitable culture medium. Such medium may be employed to maintain viability, or, in more preferred embodiments, to permit the immobilized cells to multiply by culturing the cells on the microarray. In one sub-embodiment of such a preferred embodiment, the support will contain accessible ligand-binding molecules sufficient to permit newly created cells, by virtue of their contact with the support during their formation, to become immobilized to the support. In a second sub-embodiment of such a preferred embodiment, the support will be treated to render unused ligand-binding molecules inaccessible to binding, so that newly created cells will be shed into the supernatant and not become immobilized to the support. In a third sub-embodiment of such preferred embodiment, the microarray will be formed using two or more different ligand-binding molecules, so that cells immobilized to one such ligand bonding-molecule will produce progeny that will bind to the support only if they possess a different ligand-binding molecule (i.e., unused ligand-binding molecules used to effect the binding of the initial cells will be rendered inaccessible to binding by progeny cells; the binding of such progeny cells to the microarray will depend upon their arraying of a ligand molecule capable of binding to a different ligand-binding molecule of the support).

The target cells may be derived from any of a wide variety of biological samples, especially those derived from a human or other animal source (such as, for example, blood, stool, sputum, mucus, serum, urine, saliva, semen, teardrop, a biopsy sample, a histology tissue sample, a PAP smear, a mole, a wart, an agricultural product, waste water, drinking water, milk, processed foodstuff, air, etc.) including samples derived from a bacterial or viral preparation, as well as other samples (such as, for example, agricultural products, waste or drinking water, milk or other processed foodstuff, air, etc.). The immobilized target cell may thus be eukaryotic cells (especially yeast, fungal, plant, bird, or insect) cells, or, more preferably, mammalian cells (especially human, simian, murine, rat, ovine, porcine, bovine, feline, canine, etc.), prokaryotic cells (e.g., bacteria (including pathogenic bacteria, such as B. anthracis, M. pneumoniae, S. aureus, S. typhus, E. coli, etc.), or viruses or viral particles. Target cells may comprise specific tissue types, such as lymphocytes, leukocytes, hepatocytes, stem cells, epithelial cells, monocytes, nerve cells, muscle cells, erythrocytes, etc. Additionally, through selection of a relevant bioconjugate, the invention permits the target cell to be one belonging to a sub-population of such cells (e.g., CD44⁺ monocytic cells, 61D3⁺/63D3⁺ monocyte cells, etc.). Alternatively, pure populations of cells may be obtained using techniques such as laser microdissection (Player, A. et al., “Laser Capture Microdissection, Microarrays And The Precise Definition Of A Cancer Cell,” Expert Rev Mol Diagn. November 2004;4(6):831-840).

The solid support may be employed in a variety of forms, including but not limited to microwell plates, glass slides, silk screened glass plates (Erie Scientific, New Hampshire), membranes and fibers; further, the support may be coated onto various materials (such as pipette tips, test tubes, etc.). Presently preferred for use as supports are polypropylene, polyvinylidene methacrylate and polyvinylidene fluoride. The supports may be 2-dimensional (so as to comprise a surface to which the oligonucleotide is bound), or 3-dimensional (so as to comprise a matrix in which bound oligonucleotides are embedded).

Any of a wide variety of solid supports may be employed in accordance with the principles of the present invention. Such supports may be glass, plastic, gel (agarose, acrylamide, etc.) paper, etc. (see, e.g., U.S. Pat. No. 5,445,934 (Fodor, S. P. A. et al.)); U.S. Pat. No. 5,919,523 (Sundberg, S. A. et al.)); U.S. Pat. No. 5,959,098 (Goldberg, M. et al.); U.S. Pat. No. 5,648,213 (Reddy, M. P. et al.); U.S. Patent Appln. Publn. No. 2002/0182629 (Rich, P. M.); PCT Publn. No. W02004/076678 (Green, L.); PCT Publn. No. WO2004/005477 (Charych, D. et al.)). For use in a variety of conventional assay methods, granular or pulverulent solid supports are particularly suitable. These materials typically have a particle size in the range of about 1 μm to about 1 inch. Suitable materials for preparation of this type of solid support include, but are not limited to, the following: polyvinylidene methacrylate (e.g., available commercially as Fractogel from Merck, Darmstadt, Germany and as Toyopearl from TosoHaas, Philadelphia, Pa.); polypropylene; polystyrene; glass beads; cellulosic materials, such as cellulosic filter paper (e.g., Actigel and Biobind as available commercially from Sterogene Bioseparation, Inc., Arcadia, Calif.); and polyvinylidene fluoride or PVDF (available commercially as Immobilon from Millipore, San Francisco, Calif.). Exemplary polyvinylidene methacrylate products (e.g., the aforementioned Fractogel and Toyopearl products) are hydrophilic macroporous packings well known to those working in the field as suitable for use in bioprocessing chromatography. The products are methacrylate-based supports copolymerized with polyvinyl alcohol; their methacrylic backbone structure makes the spherical beads rigid. They are stable at pH 1 to 14 and at temperatures up to 100° C., resistant to chemical attack, and not degraded by microbes. The packings are available in various pore size ranges; particularly suitable for use in accordance with the present invention are Toyopearl HW-75 and Fractogel-75F, which have a particle size of about 45 μm [“TosoHaas TSK-GEL Toyopearl,” TomHaas, Philadelphia, Pa. (March 1989)]. Other suitable materials with comparable properties would of course be readily apparent to those skilled in the art.

In one embodiment, latex microparticles, or other matrix-generating solid supports, may be employed to provide a non-planar (e.g., 3-dimensional) array. Whereas planar arrays permit the identification and characterization of binding based on the position of labeled cells or molecules on the planar array, non-planar, and especially bead-based non-planar arrays, permit the identification and characterization of binding by providing differential labeled, or detectably distinguishable, particles or beads. For example, beads having biological labels or binding ligands attached to their surfaces can be impregnated with different concentrations of dyes (e.g., fluorescent, luminescent, etc.) or labels (e.g., radioisotopic, enzymatic, etc.) and then incubated with cells or other molecules to produce a non-planar array in which the presence of a target cell, ligand, etc., is detected through the differential detection of beads that have become bound to such cells or molecules (Venkatasubbarao, S. “Microarrays—Status and Prospects,” Trends in Biotechnology December 2004 22(12):630-637; Morgan, E. et al. “Cytometric Bead Array: A Multiplexed Assay Platform With Applications In Various Areas Of Biology, Clin. Immunol. (2004) 110:252-266. Alternatively, micro- or nano-barcodes (Nicewamer-Pena, S. R. et al., “Submicrometer Metallic Barcodes,” Science. Oct. 5, 2001;294(5540): 137-41; Chan, W. C. W. et al., “Luminescent Quantum Dots For Multiplexed Biological Detection And Imaging,” Curr Opin Biotechnol. February 2002;13(l):40-6) or cylindrical nanoparticles prepared from inert metals such as gold, silver, nickel or platinum (Nanoplex, Mountain View, Calif.) may be employed. In a further embodiment, the employed particles can be of different sizes and/or internally dyed with different concentrations of dyes to identify the particles so as to permit their differential detection (Edwards, B. S. et al. “Flow Cytometry For High-Throughput, High-Content Screening, Curr. Opin. Chem. Biol. 2004 8:392-398). Luminex (Austin, Tex.) sells addressable bead arrays, containing up to 100 beads, with different ratios of fluorescent dyes. In a further embodiment, a micron-sized optical ‘imaging’ fiber may be etched into the beads so as to permit the beads to fit into wells on the tip of the fiber. Different oligonucleotide sequences may be attached to each bead and coupled to cells or other molecules in accordance with the principles of the present invention. Thousands of beads can be self-assembled on the fiber bundle. A subsequent decoding process is carried out to determine which bead occupies which well. Bound cells or molecules can be measured, for example, using a fluorescent label (Gunderson, K. L. et al. “Decoding Randomly Ordered DNA Arrays, Genome Res. 2004 14:870-877; Oliphant, A. et al., “Beadarray™ Technology: Enabling An Accurate, Cost-Effective Approach To High-Throughput Genotyping,” Biotechniques 2002 32:S56-S61; ). Additionally, micro- and nano-technology may be employed to introduce microtransponders or other integrated circuit or microcircuits (e.g., of 250 μm×250 μm×100 μm dimensions) containing for example a photocells, memory, clock, antenna, etc. Each such microtransponder, circuit or microcircuit can be unique and identifiable. Oligonucleotides or proteins can be immobilized on to such microtransponders, circuits or microcircuits to differential label, or detectably distinguish such particles (Cain, J. T. et al. “Energy Harvesting For DNA Gene Sifting And Sorting,” Intl. J. Parallel Distributed Sys. Networks 2001 4:140-149). Such non-planar arrays can be employed with detection platforms of Beckman Coulter, Inc. or Luminex. For example, the LS™ Analyzer (Beckman Coulter, Inc.) may be configured to detect optically distinguishable beads of a non-planar array. The Multisizer™, N4 Plus™, N5™, RapidVUE™ Z1™ or Z2™ (Beckman Coulter, Inc.) may be employed to distinguish beads of differing sizes.

In an alternative embodiment, the solid supports of the present invention will provide a planar array, such as the A² platform (Beckman Coulter) and the IC100 platform (Beckman Coulter, Inc.) (U.S. Pat. No.5,648,213 (Reddy, M. P). An exemplary polyvinylidene fluoride material for use in accordance with such an embodiment of the present invention is the aforementioned Immobilon AV Affinity Membrane. This product is a chemically activated, hydrophilic microporous membrane to which a variety of ligands can be covalently immobilized. The solid phase matrix offers a high capacity for covalent immobilization (>100 μg/cm²) with retention of biological activity. The base membrane material is a non-interactive polymer (hydrophilic polyvinylidene difluoride) that has low levels of non-specific protein adsorption (<1 μg/cm²). The entire external and internal surface of the membrane is chemically derivatized to allow for covalent immobilization of materials containing amino groups (“Immobilon AV Affinity Membrane,” Millipore, San Francisco, Calif. (June 1988)). Again, other comparable materials would be apparent to those working in the field.

Most preferably the supports of the present invention will be planar and composed of glass, plastic, cellulose, film, paper, etc. and will contain an ordered pattern of ligand-binding molecules (so as to form a microarray of spots or regions 10 μm -300 μm or more in diameter (most preferably 10 μm −200 μm in diameter). Examples of planar arrays can be found in U.S. Pat. Nos. 5,807,522 and 6,649,404, and in Brown, P. O. et al. (“Exploring The New World Of The Genome With DNA Microarrays,” Nat. Genet. 1999 21 (1 Suppl):33-37); Chee, M. R. et al. (Accessing Genetic Information With High-Density DNA Arrays,” Science 1996 274:610-614); Sosnowski, R. G. et al. (“Rapid Determination Of Single Base Mismatch Mutations In DNA Hybrids By Direct Electric Field Control,” Proc. Natl. Acad. Sci. USA 1997 94:1119-1123). Such surfaces may be coated (as with any of the above-indicated compositions, etc.), or may be uncoated. They may be rigid or flexible. In a highly preferred embodiment, such planar arrays will be configured as arrays within a larger array so as to be amenable for analysis using the A² platform and the IC100 platform (Beckman Coulter, Inc.) (U.S. Pat. No. 5,648,213 (Reddy, M. P).

Yet another suitable solid support material is optical waveguides. Chemical sensors consisting of optical fibers and planar waveguides bearing chemically selective immobilized reagents have the potential to be fast, sensitive and specific analytical tools. These sensors exploit the optical properties of interfaces between two transparent media having different refractive indices. Under appropriate conditions, light can propagate within an optical waveguide (such as a quartz rod immersed in an aqueous solution) by total internal reflection. As part of this process, an evanescent wave penetrates a fraction of a wavelength into the aqueous phase and can optically interact with molecules located within a thin evanescent wave zone outside the waveguide surface. In particular, fluorescent molecules bound to the fiber surface may fall within this evanescent wave zone and may be excited by the evanescent wave. An oligonucleotide covalently bound to the waveguide can be employed in accordance with the present invention to harvest immunochemical conjugate containing a fluorescent label. As a result of this process, the fluorescent labels (which are indicative of analyte concentration) are brought into the evanescent wave zone at the fiber surface and are excited by light propagating along the fiber axis. The resultant fluorescent emission is captured by the fiber and carried by total internal reflection to a detector located at the end of the fiber.

The unique advantage of applying the present invention to fiber optic detection is that the optical fiber can be regenerated by simple denaturation of the double stranded nucleic acid complex, thus making it ready for measurement of another analyte sample. By using different fluorescent molecules of distinctly different excitation and/or emission wavelengths, simultaneous multiple analyte measurements can be made. Alternatively, simultaneous measurements can also be made by coating different fibers or bundles of fibers with different oligonucleotides, each corresponding to a specific analyte. After harvesting the signals from the homogeneous phase, a particular set of fibers may be activated at a time and the fluorescence measured to determine the concentration of a particular analyte. In certain formats, e.g., the A² format, it may not be possible or practical to completely remove all previously bound antibodies. While such limitations may serve to increase the background “noise” of the assay, they do not preclude accurate analysis.

In a preferred embodiment, the immobilization of the target cell to the support is accomplished through the use of a ligand-binding molecule—oligonucleotide bioconjugate and a support-immobilized oligonucleotide. The ligand-binding component of the bioconjugate can be any of a wide array of molecules having the ability to discern and bind to a ligand molecule present on the surface of the target cell. Suitable cellular ligands can, for example, be antigenic surface proteins (e.g., blood typing proteins, stem cell markers, cancer-associated antigens, cell surface markers (e.g., CD13, CD26, etc.), antigens diagnostic of pathogenicity, etc.), receptors (especially hormone receptors (e.g., insulin receptors, growth hormone receptors, steroid hormone receptors [these are not typically surface proteins], etc.), cytokine receptors, chemokine receptors, messenger protein receptors, opiate receptors, etc.), transmembranous enzymes (e.g., tyrosine phosphatases, guanylate cyclases, serine/threonine or tyrosine kinases, serine or tyrosine phosphatases, prostaglandin H2 synthetases, sulfases, fatty acid amide hydrolases, monoamine oxidases, etc. (see, e.g., Bracey, M. H. et al., “Structural Commonalities Among Integral Membrane Proteins,” FEBS Lett. 567:159-165 (2004)), messenger proteins (Prochiantz A., Messenger Proteins: Homeoproteins, TAT And Others,” Curr. Opin. Cell Biol. August 2000;12(4):400-406), etc. The presence of the ligand may occur naturally in healthy (i.e., normal) cells or it may be associated with the presence or severity of a disease state (such as cancer, diabetes, etc.) (i.e., abnormal cells).

Since the target cell binds to the microarray through a binding of the selected ligand-binding molecule to a ligand molecule on the surface of the target cell, the selection of the ligand-binding molecule is determinative of whether a particular cell will become bound to the microarray. Suitable ligand-binding molecules are thus selected in light of the desired ligand of the target cell. Suitable ligand-binding molecules include, for example, immunoglobulins, hormones (especially, peptide hormones, such as insulin, growth hormone, etc.), immunomodulator molecules, especially cytokines (e.g., interleukins (“IL) (such as IL-1, IL-2, IL-3, IL-4, IL-5, IL-6, IL-7, IL-8, IL-9, IL-10, IL-11, IL-12, IL-13, IL14, etc.); lymphokines and signaling molecules such as erythropoiesis stimulating proteins (e.g., erythropoietin (EPO)), tumor necrosis factor (TNF), interferons, etc., growth factors, such as transforming growth factor, nerve growth factor, brain derived growth factor, neurotrophin-3, neurotrophin-4, heptaocyte growth factor, Transforming Growth Factor (TGF, TGF-β1, TGF-β2, TGF-β3, etc.), Colony Stimulating Factors (G-CSF, GM-CSF, M-CSF etc.), Epidermal Growth Factor (EGF, LIF, KGF, OSM, PDGF, IGF-I, etc.), Fibroblast Growth Factor (αFGF, βFGF, etc.) and chemokines (see, e.g., Baggiolini et al., “Human Chemokines. An Update,” Ann. Rev. Immunology 1997 15:675-705; Zlotnik et al., “Recent Advances In Chemokines And Chemokine Receptors,” Critical Rev. Immunology 1999 19(1): 1-4; Wang et al., “Chemokines And Their Role In Tumor Growth And Metastasis,” J. Immunological Methods 1998 220(1-2): 1-17; and Moser et al., “Lymphocyte Responses To Chemokines,” Intl. Rev. Immunology 1998 16(3-4):323-344), blood typing markers (e.g., A, B, Rₕ, M, N, etc.). One clear example would be blood typing-populating the microarray with oligonucleotide conjugates of antibodies specific for markers for the various blood typing markers on the surfaces of red blood cells (A, B, Rh, M, N, etc.) (see, e.g., Storry, J. R., “Human Blood Groups: Inheritance And Importance In Transfusion Medicine,” J. Infus. Nurs. 2003 26(6):367-372; Oriol, R., “Molecular Genetics of H,” Vox Sang. 2000 78 Suppl 2:105-108; Ikemoto, S., “Searching For Genetic Markers—In The Fields Of Forensic Medicine And Human Genetics,” Nippon Hoigaku Zasshi. 1995 49(6):419-431; Cartron, J. P. et al., “Red Cell Membrane Diseases And Blood Group Abnormalities,” Rev Fr Transfus Immunohematol. 1983 26(6):599-623; Gohler, W. (1981) “The Importance Of Serogenetics As A Subspecialty Of Human Genetics,” Z Gesamte Inn Med. 36(11):342-347); tumor-specific or tumor-associated markers, pharmacological agents having the ability to bind to cellular ligands (especially pharmacological agents that bind to cellular ligands involved in signal transduction), substrates or inhibitors of transmembranous enzymes, etc.

As used herein, the term “immunoglobulin” includes natural or artificial mono- or polyvalent antibodies and polyclonal and monoclonal antibodies, and also molecules that are fragments and derivatives of such, including, for example, F(ab′)₂, Fab′ and Fab fragments, chimeric antibodies, hybrid antibodies having at least two antigen or epitope binding sites, single polypeptide chain antibodies, bispecific recombinant antibodies (e.g. quadromes, triomes), interspecies hybrid antibodies, and molecules that have been chemically modified and must be regarded as derivatives of such molecules and which may be prepared either by the known conventional methods of antibody production or by DNA recombination, using hybridoma techniques or antibody engineering or synthetically or semisynthetically in known manner. Methods for isolating or obtaining immunoglobulins are well-known in the art (Kohler, G. et al., “Continuous Cultures Of Fused Cells Secreting Antibody Of Predefined Specificity,” Nature 1975 256:495-497; Taggart, R. T. et al., Stable Antibody-Producing Murine Hybridomas,” Science 1983219:1228-1230; Kozbor, D. et al., Immunology Today 1983 4:72-79; Morrison et al., “Chimeric Human Antibody Molecules: Mouse Antigen-Binding Domains With Human Constant Region Domains,” Proc. Natl. Acad. Sci. USA 198481:6851-6855; Takeda, S. et al., “Construction Of Chimaeric Processed Immunoglobulin Genes Containing Mouse Variable And Human Constant Region Sequences,” Nature 1985 314:452-454; Biocca, S. et al., “Expression And Targeting Of Intracellular Antibodies In Mammalian Cells,” EMBO J. 1990 9:101-108; Bird, R. E. et al., “Single-Chain Antigen-Binding Proteins,” Science 1988 242:423-426; Boss, M. A. et al., “Assembly Of Functional Antibodies From Immunoglobulin Heavy And Light Chains Synthesised In E. coli,” Nucl. Acids Res. 1984 12:3791-3806; Boulianne, G. L. et al., “Production Of Functional Chimaeric Mouse/Human Antibody,” Nature 1984 312:643-446; Bukovsky, J. et al. “Simple And Rapid Purification Of Monoclonal Antibodies From Cell Culture Supernatants And Ascites Fluids By Hydroxylapatite Chromatography On Analytical And Preparative Scales,” Hybridoma 1987 6:219-228; Diano, M. et al., “A Method For The Production Of Highly Specific Polyclonal Antibodies,” Anal. Biochem. 1987 166:224-229; Huston J. S. et al., “Protein Engineering Of Antibody Binding Sites: Recovery Of Specific Activity In An Anti-Digoxin Single-Chain Fv Analogue Produced In Escherichia coli,” Proc. Natl. Acad. Sci. USA 1988 85:5879-5883; Jones, P. T. et al., “Replacing The Complementarity-Determining Regions In A Human Antibody With Those From A Mouse,” Nature 1986 321:522-525; Langone, J. J. et al. (Eds.), Methods Enzymol. 1987 121, Academic Press, London; Oi, V. T. et al., BioTechniques 1986 4:214-221; Riechmann, L. et al., “Reshaping Human Antibodies For Therapy,” Nature 1988 332:323-327; Tramontano, A. et al., “Chemical Reactivity At An Antibody Binding Site Elicited By Mechanistic Design Of A Synthetic Antigen,” Proc. Natl. Acad. Sci. USA 1986 83:6736-6740; Wood, C. R. et al., “The Synthesis And In Vivo Assembly Of Functional Antibodies In Yeast,” Nature 1985 314:446-449; and U.S. Pat. No. 4,946,778 (Ladner, R. et al.)).

Polyclonal antibodies may be produced through any of a variety of well known methods. For example, various animals may be immunized for this purpose in known manner by injecting them with an antigen (for example, the target biological molecule, or another molecule sharing an epitope of the target biological molecule. Such antigen molecules may be of natural origin or obtained by DNA recombination or synthetic methods, or fragments thereof and the desired polyclonal antibodies are obtained from the resulting sera and purified by known methods. Alternatively, intact cells that array the target biological molecule may be used. Various adjuvants may also be used for increasing the immune response to the administration of antigen, depending on the animal selected for immunization. Examples of these adjuvants include Freund's adjuvant, mineral gels such as aluminum hydroxide, surfactant substances such as polyanions, peptides, oil emulsions, haemocyanins, dinitrophenol or lysolecithin.

If desired, the ligand-binding molecule of the bioconjugates of the present invention may be purified to achieve a desired degree of purity. Methods for accomplishing such purification are well known to those of ordinary skill (e.g. by immunoabsorption or immunoaffinity chromatography, by HPLC (High Performance Liquid Chromatography) or combinations thereof). Suitable antibody fragments may also be prepared by known methods. For example, F(ab′)₂ fragments may be obtained by pepsin digestion of the complete polyclonal or monoclonal antibody. Fab′ fragments may be obtained by reducing the disulfide bridges of the associated F(ab′)₂ fragment, for example, and Fab fragments may be obtained, for example, by treating the antibody molecules with papain and a reducing agent.

In a preferred embodiment, the oligonucleotide linker system comprises a first oligonucleotide that is covalently bonded to the ligand-binding molecule and a second oligonucleotide that is immobilized to the solid support. It is preferred that the oligonucleotides have a length of at least 6 bases, preferably about 10 bases, more preferably at least about 20 bases, and most preferably about 30 bases. As is well understood in the art, the strength of the duplexes formed is determined to some extent by the sequence composition of the pair of oligonucleotides; in particular, stable duplexes may be formed with short (i.e., 6-10 base) oligomers using, e.g., modified bases or peptide nucleic acids.

Suitable oligonucleotides may be produced by any of a variety of methods: synthetically (see, e.g., Herdewijn, P. “Oligonucleotide Synthesis Methods and Applications” Humana Press, Totowa, N.J. (2004); Caruthers, M. H. “Chemical Synthesis Of DNA And DNA Analogues,” Acc. Chem. Res. 24, 278-284 1991); Beaucate, S. L., et al. “Synthesis Of Oligonucleotides” Tetrahedron, 48:2290-2291 1992; Wright, P. et al. “Large Scale Synthesis of Oligonucleolides via Phosphoramidte Nucleosides and a High-Loaded Polystyrene Support,” Tetrahedron Letters, 34(21):3373-3376 1993; Wolter, A., et al; “Polymer Support Oligonucleotide Synthesis XX.sup.1: Synthesis of a Henhectacosa Deoxynucleotide By Use of a Dimeric Phosphoramidite Synthon”; Nucleosides & Nucleosides, 5(1), pp. 65-77 (1986); obtained from naturally occurring nucleic acid molecules (e.g., by digestion of DNA with one or more restriction endonucleases), or produced through the use of recombinant technology (e.g., cloning, DNA amplification technologies (e.g., PCR (Mullis, K. et al., U.S. Pat. No.4,683,202) rolling circle amplification (U.S. Pat. No. 6,740,745 (Auerbach, J. I.)), etc.).

The sequences of the first and second oligonucleotides are selected so as to permit them to hybridize to one another. Homopolymer molecules (i.e., in which one oligonucleotide is poly dA and the other is poly dT, or in which one oligonucleotide is poly dC and the other is poly dG) may be employed, however, heteropolymer molecules (in which the sequences of the oligonucleotide contain 2, 3 or 4 different nucleotide species) work quite efficiently.

Heteropolymer molecules permit the detection of multiple ligands in a single sample through the use of specific pairs of sequences for harvesting and displacement of each analyte. Suitable oligonucleotides include, but are not limited to, those comprising conventional DNA and RNA bases, DNA/RNA base analogs (see, e.g., Frauendorf, A. et al., “Studies in Natural Products Chemistry,” 13, 257 (1993); Milligan, J. et al., “Current Concepts In Antisense Drug Design,” J. Medicinal Chem. 36, 1923 (1993)) and peptide nucleic acids (PNAs) (see, e.g., Hanvey, J. C. et al., “Antisense And Antigene Properties Of Peptide Nucleic Acids,” Science 258, 1481 (1992); Burchardt, O. et al., Trends in Biotechnology 11, 384 (1993)). In general, any oligonucleotides capable of base pairing (e.g., forming a Watson-Crick duplex or a Hoogstein triplex complex) would be suitable for use in accordance with the invention. The oligonucleotides may be single-stranded or partially or fully double-stranded, but are preferably single stranded. They may comprise naturally occurring nucleotide residues or modified or synthetic nucleotide residues.

In general, it is further preferred that the oligonucleotide pairs be completely complementary over at least a portion of their respective sequences. These complementary portions of the sequences should comprise at least 6 bases, preferably at least about 10 bases, more preferably at least about 20 bases, and most preferably at least about 30 bases. Of course, as is well understood in the art, using appropriate low-stringency conditions it is possible to achieve hybridization even when a limited degree of mismatch exists between the two oligonucleotides. Nonetheless, for purposes of convenience, the use of completely complementary sequences is preferred. In general, the amount of oligonucleotide bound to the support is in excess of the oligonucleotide of the bioconjugate component of the present invention.

### II. GENERATION OF THE MICROARRAYS OF THE INVENTION

### A. Immobilization of the Support-Immobilized Oligonucleotide to the Support

The oligonucleotides may be immobilized to the support using any of a variety of techniques (see, e.g., U.S. Pat. No. 5,648,213 (Reddy, M. P. et al.), Yeo, D. S. et al., “Strategies For Immobilization Of Biomolecules In A Microarray,” Comb. Chem High Throughput Screen. May 2004;7(3):213-221; U.S. Pat. No.6,747,143 (Stryer, L. et al.), etc.). Pursuant to one approach, described in U.S. Pat. No.5,648,213 (Reddy, M. P. et al.), the oligonucleotide is synthesized directly on the support in a manner as conventionally employed in the synthesis of oligonucleotides for other purposes; both particulate and membrane supports may be suitably employed as a substrate for oligonucleotide synthesis. Alternatively, an oligonucleotide containing a reactive functionality (e.g., an amino or thiol group) may be immobilized onto a support containing a suitable functionality reactive therewith, forming a covalent bond between the oligonucleotide and the support. Yet another approach involves attachment of the oligonucleotide to the support by affinity binding; for example, a biotinylated oligonucleotide may be immobilized onto a support containing avidin or streptavidin. As would be apparent to those working in the field other techniques may equally well be employed to attach the oligonucleotide to the support.

### B. Production of the Ligand-Binding Moleucle-Oligonucleotide Bioconjugate

In a preferred embodiment of the invention, the microarrays comprise a bioconjugate of an oligonucleotide and a protein ligand-binding molecule. The conjugation of the oligonucleotide and a protein ligand-binding molecule may be accomplished by any of a variety of means.

Any of a variety of different coupling chemistries may be employed. Pursuant to one approach, a homobifunctional agent (for example, 1,4-phenylene diisothiocyanate) is employed. Suitable conjugates may also be prepared by glutaraldehyde crosslinking, maleimide-thiol coupling (Ghosh, S. S. , et al., “Use Of Maleimide-Thiol Coupling Chemistry For Efficient Syntheses Of Oligonucleotide-Enzyme Conjugate Hybridization Probes,” Bioconjug. Chem. January-February 1990;1(1):71-6), isothiocyanate-amine coupling (Brandtzaeg, P. “Conjugates Of Immunoglobulin G With Different Fluorochromes. I. Characterization By Anionic-Exchange Chromatography,” Scand. J. Immunol. 2: 273-290 1973; Loken, M. R. et al., “Analysis Of Cell Populations With A Fluorescence-Activated Cell Sorter,” 1975 Annals N.Y. Acad. Sci. 254: 163-171; U.S. Pat. No.5,648,213 (Reddy, M. P. et al.); Keller, G. H., et al., “DNA Probes,” MacMillan Publishers Ltd., 1989), and Schiff base formation/reduction.

In circumstances in which the ligand-binding molecule possesses a suitable thiol group, a more preferred conjugation approach involves the use of a heterobifunctional reagent as disclosed in U.S. Pat. No.5,648,213 (Reddy, M. P. et al.). Suitably, such a reagent includes a first reactive group (e.g., N-hydroxysuccinimide) specific for amino groups of the oligonucleotide and a second reactive group (e.g., maleimide) specific for thiol groups of the antibody or fragment thereof. The use of such heterobifunctional reagents provides substantially higher yields; whereas a homobifunctional agent may react with any of the multiple amino groups of an antibody or fragment thereof as well as the oligonucleotide (and thus, lead to a mixture of products), a suitable heterobifunctional reagent reacts specifically to form a one-to-one antibody/oligonucleotide conjugate. As Fab′ fragments have only one thiol group, they are particularly suitable for use in formation of conjugates with oligonucleotides using this method. Moreover, Fab′-oligonucleotide conjugates often give superior results in immunoassays in accordance with the present invention relative to whole antibody-oligonucleotide conjugates, particularly in competitive binding assays. This may be rationalized by the fact that a Fab′ fragment has only one binding region for the hapten or analyte, and hence provides greater sensitivity in the competitive binding reaction compared to the whole antibody (which has two binding regions for the hapten or analyte). One preferred heterobifunctional agent for use in preparation of antibody/oligonucleotide conjugates is N-sulfosuccinimidyl 4-(N-maleimidomethyl)cyclohexane-1-carboxylate (Sulfo-SMCC).

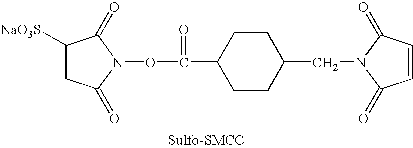

As would be readily appreciated by those skilled in the art, however, a variety of amino and sulfhydryl group directed cross-linkers can equally well be employed in accordance with the principles of the present invention. Such cross-linkers are described, for example, in Wong, S. S., “Chemistry of Protein Conjugation and Cross-linking,” CRC Press, Boca Raton, Fla. (1 991), pp. 147-164. Exemplary cross-linking agents of this type include the following: N-succinimidyl 3-(2-pyridyidithio)propionate; N-succinimidyl maleimidoacetate; N-succinimidyl 3-maleimidopropionate; N-succinimidyl 4-maleimidobutyrate; N-succinimidyl 6-maleimidocaproate; N-succinimidyl 4-(N-maleimidomethyl)cyclohexane-1-carboxylate; N-succinimidyl 4-(p-maleimidophenyl)butyrate; N-sulfosuccinimidyl 4-(p-maleimidophenyl) butyrate; N-succinimidyl o-maleimidobenzoate; N-succinimidyl m-maleimidobenzoate; N-sulfosuccinimidyl m-maleimidobenzoate; N-succinimidyl p-maleimidobenzoate; N-succinimidyl 4-maleimido-3-methoxybenzoate; N-succinimidyl 5-maleimido-2-methoxybenzoate; N-succinimidyl 3-maleimido-4-methoxybenzoate; N-succinimidyl 3-maleimido-4-(N,N-dimethyl)aminobenzoate; maleimidoethoxy[p-(N-succinimidylpropionate)phenoxy]ethane; N-succinimidyl4-[(N-iodoacetyl)amino]benzoate; N-succinimidyl 3-maleimido-4-(N,N-dimethyl)aminobenzoate; maleimidoethoxy[p-(N-succinimidylpropionate)-phenoxy]ethane; N-succinimidyl4-[(N-iodoacetyl)amino]benzoate; N-sulfosuccinimidy 4-[(N-iodoacetyl)amino]benzoate; N-succinimidyliodoacetate; N-succinimidylbromoacetate; N-succinimidyl3-(2-bromo-3-oxobutane-1-sulfonyl)propionate; N-succinimidyl 3-(4-bromo-3-oxobutane-1-sulfonyl)propionate; N-succinimidyl 2,3-dibromopropionate; N-succinimidyl 4-[(N,N-bis(2-chloroethyl)amino]phenylbutyrate; p-nitrophenyl 3-(2-bromo-3-oxobutane-1-sulfonyl)propionate; p-nitrophenyl-3-(4-bromo-3-oxobutane-1-sulfonyl)propionate; p-nitrophenyl 6-maleimidocaproate; (2-nitro-4-sulfonic acid-phenyl)-6-maleimidocaproate; p-nitrophenyliodoacetate; p-nitrophenylbromoacetate; 2,4-dinitrophenyl-p-(β-nitrovinyl)benzoate; N-3-fluoro-4,6-dinitrophenyl)cystamine; methyl 3-(4-pyridyidithio)propionimidate HCl; ethyl iodoacetimidate HCl; ethyl bromoacetimidate HCl; ethyl chloroacetimidate HCl; N-(4-azidocarbonyl-3-hydroxyphenyl)maleimide; 4-maleimidobenzoylchloride; 2-chloro-4-maleimidobenzoyl chloride; 2-acetoxy-4-maleimidobenzoylchloride; 4-chloroacetylphenylmaleimide; 2-bromoethylmaleimide; N-[4-{(2,5-dihydro-2,5-dioxo-3-furanyl)methyl}thiophenyl]-2,5-dihydro-2,5-dioxo-1H-pyrrole-1-hexanamide; epichlorohydrin; 2-(p-nitrophenyl)allyl-4-nitro-3-carboxyphenylsulfide; 2-(p-nitrophenyl)allyltrimethylammonium iodide; α,α-bis[{(p-chlorophenyl)sulfonyl}methyl]acetophenone; α,α-bis[{(p-chlorophenyl)sulfonyl}methyl]-p-chloroacetophenone; α,α-bis[{(p-chlorophenyl)sulfonyl}methyl]-4-nitroacetophenone; α,α-bis[(p-tolylsulfonyl)methyl]-4-nitroacetophenone; α,α-bis[{(p-chlorophenyl)sulfonyl}methyl]-m-nitroacetophenone; α,α-bis[(p-tolylsulfonyl)methyl]-m-nitroacetophenone; 4-[2,2-bis{(p-tolylsulfonyl)methyl}acetyl]benzoic acid; N-[4[2,2-2{(p-tolylsulfonyl)methyl}acetyl]benzoyl]-4-iodoaniline; α,α-bis[(p-tolylsulfonyl)methyl]p-aminoacetophenone; N-[{5-(dimethylamino)naphthyl}sulfonyl]α,α-bis[(p-tolylsulfonyl)methyl]-p-aminoacetophenone; and N-[4-{2,2-bis(p-tolylsulfonyl)methyl}acetyl]benzoyl-1-(p-aminobenzyl)dieth ylenetriaminepentaacetic acid.

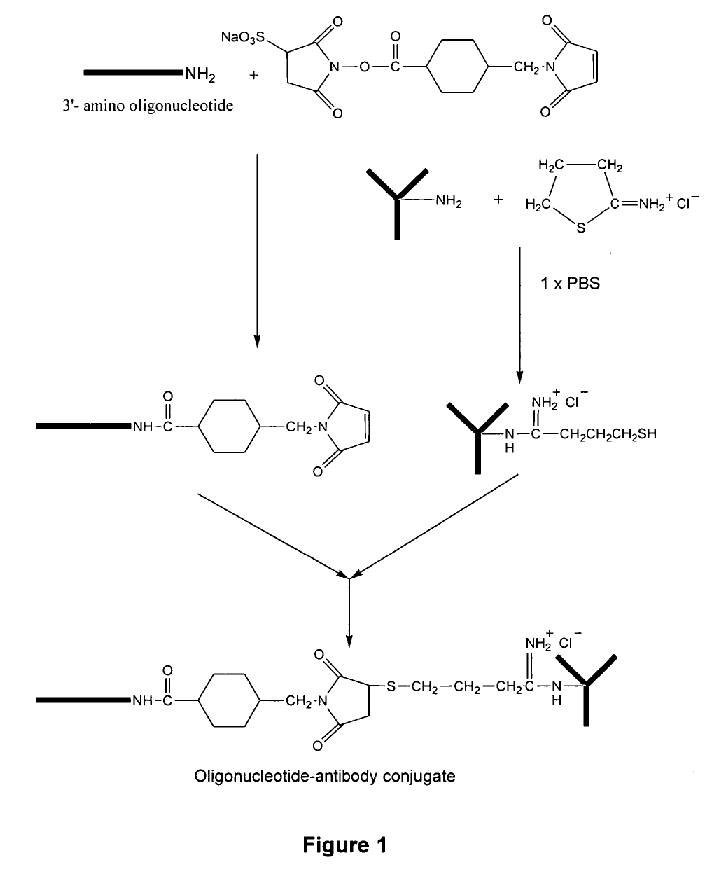

Alternatively, and more preferably, formation of the bioconjugate can be accomplished through the covalent coupling of a thiolated amino group of the ligand-binding molecule with an aminated group of the oligonucleotide. In accordance with such an embodiment, the synthesis of the oligonucleotide-protein conjugate is accomplished in four steps (FIG. 1):

1. Synthesis of an oligonucleotide having an amino group;

2. Activation of the 3′ amino group by a heterofunctional linker;

3. Thiolation of an amino group of the protein to be coupled; and

4. Coupling of the activated oligonucleotide and the thiolated protein.

The amino group of the oligonucleotide may be present at the 3′ terminal residue of the oligonucleotide, at the 5′ terminal residue of the oligonucleotide, or at a site between the termini of the molecule (i.e., an internal site).

The synthesis of an oligonucleotide having an amino group is preferably accomplished using an amino modifier reagent such as C7 CPG (Glen Research, Sterling Va.):

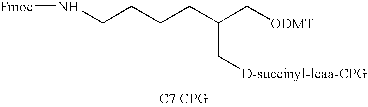

In order to link the modified oligonucleotide to the protein, a heterofunctional linker is employed. Preferably, such heterofunctional linker will have one moiety (such as an NHS-ester moiety) that is able to react with the primary amine of the 3′amino oligonucleotide and a second moiety (such as a maleimide group) that is capable of reacting with a thiol group. Examples of suitable heterofunctional linkers include:

| | |
|---|---|
| Sulfo-SMCC | sulfosuccinimidyl 4-(N-maleimidomethyl)cyclohexane- |
| | 1-carboxylate |
| Sulfo-EMCS | N-(ε-Maleimidocaproyloxy) sulfosuccinimide ester |
| Sulfo-GMBS | (N-(γ-Maleimidobutyryloxy) sulfosuccinimide ester |
| Sulfo-KMUS | N-(K-Maleimidoundecanecanoyloxy) sulfosuccinimide |
| | ester |
| Sulfo-LC-SPDP | sulfosuccinimidyl 6-(3′-(2-pyridyldithio)- |
| | propionamido)hexanoate |
| Sulfo-MBS | m-Maleimidobenzoyl-N-hydroxysulfosuccinimide ester |
| Sulfo-SIAB | sulfosuccinimidyl(4-iodoacetyl)aminobenzoate |
| Sulfo-SMPB | sulfosuccinimidyl 4-(p-maleimidophenyl)butyrate |
| Sulfo-LC-SMPT | sulfosuccinimidyl-6-(α-methyl-α-(2- |
| | pyridyldithio)toluamido)hexanoate |
| SVSB | (N-succinimidyl-4-vinylsulfonyl)benzoate |
| SIA | N-succinimidyl iodoacetate or iodoacetic acid N- |
| | hydroxysuccinimide ester |
| SIACX | (succinimidyl 6-(4-iodoacetyl)amino methyl- |
| | cyclohexane-1-carbonyl)amino hexanoate |
| SIAXX | succinimidyl 6(6-(((iodoacetyl)amino |
| | hexanoyl)aminohexanoate)) |
| NPIA | p-nitrophenyl iodoacetate |

All of these reagents can be purchased from Pierce, Rockford, Ill.). Sulfo-SMCC (sulfosuccinimidyl 4-(N-maleimidomethyl)cyclohexane-1-carboxylate) is a preferred heterofunctional linker. The use of this compound is described by Samoszuk, M. K., et al., (“A peroxide-generating immunoconjugate directed to eosinophil peroxidase is cytotoxic to Hodgkin's disease cells in vitro,” Antibody, Immunoconjugates Radiopharmaceuticals 2(1), 37-45 (1989)).

Preferably, the linker is reacted with the amino oligonucleotide prior to its reaction with the protein. Reaction at a pH of approximately 8.0-8.5 at room temperature results in the formation of an amide bond between the amino group of the oligonucleotide and the ester carbon of the linker (FIG. 1).

The amino group of the ligand-binding molecule that is to be conjugated to the oligonucleotides is preferably reacted with iminothiolane (Traut's reagent). Reaction at room temperature results in the formation of a thiol modification to the involved amino group (FIG. 1). The amino group may be the amino terminal amino group, or it may be an internal amino group (e.g., the ε amino group of a lysine or arginine residue).

Coupling between the activated oligonucleotide and the thiolated protein is preferably accomplished by mixing the thiolated protein with the sulfo-SMCC-modified oligonucleotide. Such mixing may be conducted in phosphate buffered saline (PBS), 3 M NaCl, 2 mM EDTA.

Any protein bearing a free primary amino group can be conjugated in accordance with the methods of the present invention. Such proteins include enzymes, hormones, solubilized receptor proteins, peptides, immunoglobulins, etc.

Such a procedure is simpler, and provides higher yields than the method of Rajur, S. B. et al., in which a thiol group is introduced at the 5′ or 3′ end of oligonucleotide and the thiolated oligonucleotide is then reacted with a protein via disulfide bond conjugation chemistry (Rajur, S. B. et al., Covalent Protein-Oligonucleotide Conjugates For Efficient Delivery Of Antisense Molecules Bioconjugate Chemistry 8:935-940 (1997)). It is likewise superior to the method of Hendrickson et al., in which a 5′ amino oligonucleotide (activated with N-succinimidyl thioacetate) is conjugated to an antibody that has been derivatized with sulfo-SMCC (Hendrickson, R. E. et al., “High Sensitivity Multianalyte Immunoassay Using Covalent DNA-Labeled Antibodies And Polymerase Chain Reaction,” Nucl., Acids Res. 23:522-529 (1994)), as well the method of U.S. Pat. No. 5,648,213 (Reddy, M. P. et al.) and Keller, G. H. et al., “DNA Probes,” MacMillan Publishers, Ltd. 1989).

### C. Microarray Assembly

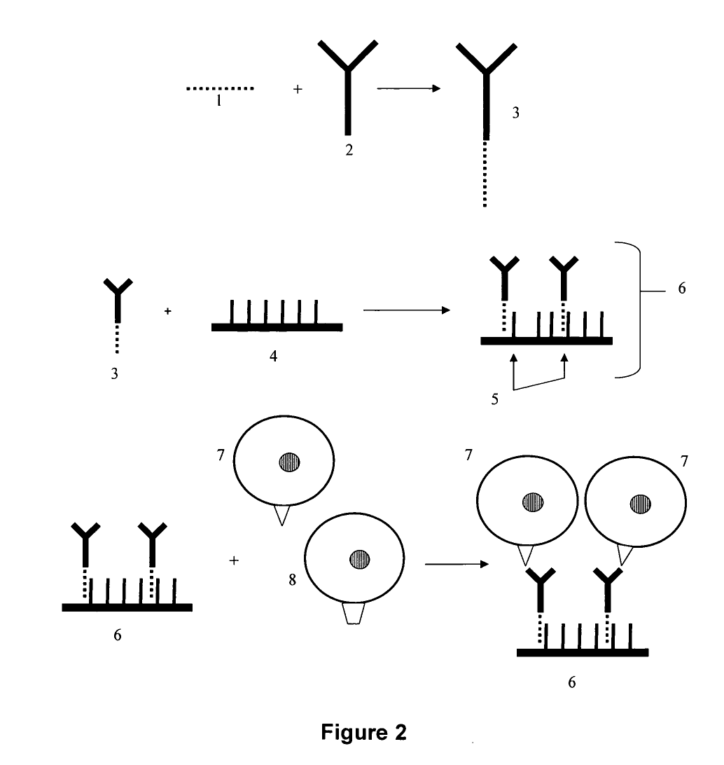

In preferred embodiments, the microarray is formed by permitting the oligonucleotide of the bioconjugate to hybridize to the support-immobilized oligonucleotide, and permitting the ligand-binding molecule of the bioconjugate to tether a cell to the support by binding to a ligand on the surface of the cell (FIG. 2). However, it will be understood that the order with which the components of the microarray are assembled is unimportant. Thus, for example, suitable microarrays can be prepared by binding a target cell with the bioconjugate and then immobilizing the cell-bioconjugate to the microarray.

The determination of optimum conditions for formation of a duplex between the complementary oligonucleotides employed in accordance with the present invention may be determined empirically in an essentially routine manner. In general, as is well known in the art, the presence of a salt (e.g., NaCl, KCl, NH₄Cl, quaternary ammonium salts, etc.) at a concentration of about 0.1M to about 3M is preferred to facilitate hybridization. In particular, the temperature at which 50% duplex formation for a pair of complementary oligonucleotides (referred to as the melting temperature, or Tₘ) occurs may be routinely determined for any given pair of oligonucleotides. The Tₘ is dependent upon a number of factors, including the length and composition of the sequences and the binding affinity of the particular bases employed in the oligonucleotides. For any given pair of complementary oligonucleotides, the Tₘ may be routinely determined spectrophotometrically by varying the temperature and measuring the absorbance at a particular wavelength (e.g, 254 nm). Once the Tₘ is determined for any pair of oligonucleotides, it is generally desirable to use a temperature below the Tₘ so as to obtain greater than 50% binding. In general, duplex formation occurs at a temperature within the same range as complex formation; to increase the amount of duplex formation, lower temperatures are preferred.

Formation of a suitable concentration of immobilized cells may occur as quickly as in a few seconds or require as long as 24 hours; preferably, the completion of the formation of the microarray requires less than about 6 hours, and most preferably less than about 3 hours. Microarray formation may also occur over a wide range of temperatures, which is limited at the upper end by the denaturing temperature of the hybridized oligonucleotides or the dissociation temperature of the ligand binding molecule—ligand interaction, or, if viable cells are desired, by the thermal viability limit of the target cell employed. Such temperature is generally in the range of about 15° C. to about 40° C., and most preferably (for purposes of convenience) at about room temperature to about 37° C.

The pH may also be varied over a fairly broad range, with the limiting factor again being cellular viability, nucleic acid denaturation, or dissociation of the ligand binding molecule—ligand interaction. Preferably, the pH will be in the range of about 4 to about 10, and most preferably close to 7. As is well known in the art, the addition of various materials, such as horse or fetal calf serum proteins, may be useful to keep materials in solution and minimize non-specific interactions; such additives, however, are not critical. Complex formation is typically carried out in aqueous solution, optionally containing up to about 25% of a suitable non-aqueous component (e.g., alcohol, ether, glycol, etc.).

The support and materials bound thereto may then be physically separated from the solution containing unbound materials. Any reagents which are non-specifically associated with the support but have not formed a complex may then be easily removed, for example, by gentle rinsing of the support. Use of appropriate conditions (e.g., a suitable salt concentration in the rinse solution) during the rinsing step is appropriate to ensure that any duplexes formed are not prematurely dissociated.

In one embodiment, the microarray will contain (or be adapted to contain) only a single immobilized bioconjugate, and will thus permit the binding of only those cells that possess a ligand recognized by the ligand-binding molecule of the bioconjugate.

In an alternative embodiment, the microarray will contain (or be adapted to contain) 2 or more different immobilized bioconjugate molecules. Preferably, such arrays will contain (or be adapted to contain) 2-10 immobilized bioconjugate molecules, more preferably 10-100, still more preferably, 100-1000 or more immobilized bioconjugate molecules. Such microarrays may be configured so as to produce a plurality of discreet regions or zones, each containing (or adapted to contain) a different bioconjugate. The use of such microarrays permits multiple cell types or multiple ligands to become bound to the same microarray so that they can be separately assayed or studied. In one embodiment, such regions or zones will be in communication with one another, such that non-immobilized cells or reagents can be in contact with multiple regions or zones. Alternatively, such zones may be physically separated (e.g., the wells of a microtiter plate).

In an alternative embodiment, the microarray will contain (or be adapted to contain) 2 or more different immobilized bioconjugate molecules that will be randomly or pseudo randomly configured so as to produce a single region capable of containing (or adapted to contain) multiple different bioconjugates. The use of such microarrays permits multiple cell types or multiple ligands to become bound to the same microarray in a manner that enables assays or studies of cell-cell communication to be conducted.

In one embodiment, the extent of immobilization of target cells to the microarray may be determined by counting the number of immobilized cells per microarray or per region or zone. Alternatively, such extent may be determined indirectly by measuring the evolution of a cellular product or the consumption of a cellular substrate. In a preferred embodiment, however, the extent of cellular immobilization may be determined through the use of a detectably labeled binding reagent. In one embodiment, such a binding reagent will be selected for its ability to bind to a ligand of the immobilized cell (the bound ligand may be the same or different from the ligand recognized by the ligand-binding molecule of the bioconjugate).

The binding reagent may be any reagent capable of binding to a target cell. Such reagents may, for example be antibodies or antibody fragments, or any of the above-mentioned classes of ligand-binding molecules. Most preferably the binding reagent will be an antibody or antibody fragment that immunologically recognizes a surface protein or antigen of the target cell. The detectable label of such reagents may be enzymatic, colored, fluorescent, chemiluminescent, radioactive, etc.). The label may also comprise the use of biotinylated molecules, or cells, which can be detected using a streptavidin-bound fluor, such as PBXL (Zoha, S. J. et al., “PBXL Fluorescent Dyes for Ultrasensitive Direct Detection,” J. Fluor. 1999 9(3):197-208).

Preferred enzymatic labels include alkaline phosphatase, β-galactosidase, horseradish peroxidase, luciferase, urease, etc., for which chromogenic or fluorogenic substrates exist are particularly preferred. Suitable substrates for peroxidase include: TMB (3,3′,5,5′ tetramethyl-benzidine), DAB (3,3′,4,4′ diaminobenzidine), and 4CN (4-chloro-1-naphthol), which produce insoluble products. Also suitable are TMB (dual function substrate), ABTS (2,2′-azino-di[3-ethylbenzthiazoline]sulfonate), and OPD (o-phenylenediamine), which produce soluble products. Suitable substrates for alkaline phosphatase include: ELF 97 (Molecular Probes, Oregon), BBT (2′-[2-benzthiazoyl]-6′-hydroxy-benzthiazole), 1,2-dioxetane chemiluminescent substrates, BCIP/NBT (5-bromo-4-chloro-3-indolyl phosphate/nitroblue tetrazolium), and p-NPP (p-nitrophenylphosphate). Suitable substrates for horse radish peroxidase include: Amplex™ Red reagent (10-acetyl-3,7-dihydroxyphenoxazine; Molecular Probes, Oregon), guaiac, 2-2′-azino-bis(3-ethyl-benthiazoline-6-sulfonic acid), tetramethylbenzidine, phenol, 4-aminoantipyrine, and 4,5-dihydroxynaphthalene-2,7-disulfonic acid (see also U.S. Pat. Nos. 6,251,621; 5,316,906; 5,443,986; and EP 0,641,351). In lieu of chromogenic substrates, enzymatic reactions may be followed by other means (changes in pH, production of product, etc.). Although such enzymes are preferred, other enzymes can be similarly exploited, and a wide variety of chromogenic or fluorogenic substrates can be employed. For example, the carboxy terminus of single amino acids and short peptides can be conjugated to certain amine-containing fluorophores (e.g., rhodamine 110 (R110), etc.) to create fluorogenic peptidase substrates (Lucas, et al., (U.S. Pat. No. 5,698,411) and Landrum et al., (U.S. Pat. No.5,976,822)). In addition 7-aminocoumarins (AMC) can be employed to form UV light-excitable substrates (e.g., CBZ-L-phenylalanyl-L-arginine amide of AMC) for serine proteases, including cathepsins, kallikrein and plasmin. The fluorogenic t-BOC-Leu-Met-CMAC substrate can be used to measure calpain activity. Many such substrates are commercially available (Molecular Probes, Inc.).

Alternatively, radioisotopic labels, fluorescent or fluorogenic labels, colorimetric labels, paramagnetic labels, materials used as colored particles, latex particles, colloidal metals such as selenium and gold, and dye particles (see U.S. Pat. Nos. 4,313,734; 4,373,932, and 5,501,985) may be employed. Suitable chemiluminescent moieties include acridinium esters, ruthenium complexes, met al complexes (e.g., U.S. Pat. Nos. 6,281,021; 5,238,108 and 5,310,687), oxalate ester-peroxide combination, etc.). Suitable colorimetric moieties include thiopeptolides, anthroquinone dyes, 2 methoxy 4(2 nitrovinyl)phenyl β-2 acetamido 2 deoxy β D glucopyranoside; ammonium 5[4(2 acetamido 2 deoxy β D glucopyranosyloxy)3 methoxy phenylmethylene]2 thioxothiazolin 4 one 3 ethanoate hydrate; 4{2[4(β D glucosyl pyranosyloxy)3 methoxy phenyl]vinyl}1 methylquinolinium iodide, 2 methoxy 4(2 nitrovinyl)phenyl β D galactopyranoside, 2{2[4(β D galactopyranosyloxy)3 methoxyphenyl]vinyl}1 methyl quinolinium iodide, 2{2[4(β D galactopyranosyloxy)3 methoxyphenyl]vinyl}3 methyl benzothiazolium iodide, 2{2[4(β D glucopyranosyloxy)3 methoxyphenyl]vinyl}1 methyl quinolinium iodide, 2{2[4(β D glucopyranosyloxy)3 methoxyphenyl]vinyl}1 propyl quinolinium iodide, 2 {2[4(β D glucopyranosyloxy)3 methoxyphenyl]vinyl}3 methyl benzothiazolium iodide, ammonium 5[4 β D glucopyranosyloxy)3 methoxy phenylmethylene]2 thioxothiazolin 4 one 3 ethanoate hydrate, 2 methoxy 4(2 nitrovinyl)phenyl acetate, 2 methoxy 4(2 nitrovinyl)phenyl propionate, 5[4 propanoyloxy)3,5 dimethoxy phenylmethylene]2 thioxothiazolin 4 one 3 ethanoate, 5[4 butanoyloxy)3,5 dimethoxy phenylmethylene]2 thioxothiazolin 4 one 3 ethanoate, 5[4 decanoyloxy)3,5 dimethoxy phenylmethylene]2 thioxothiazolin 4 one 3 ethanoate, 5[4 dodecanoyloxy)3,5 dimethoxy phenylmethylene]2 thioxothiazolin 4 one 3 ethanoate, 5[4 tetradecanoyloxy)3,5 dimethoxy phenylmethylene]2 thioxothiazolin 4 one 3 ethanoate, Pyridinium 4{2[4(phosphoroyloxy)3,5 dimethoxyphenyl]vinyl}1 propyl quinolinium iodide, Pyridinium 5(4 phosphoryloxy 3,5 dimethoxy phenylmethylene)3 methyl 2 thioxothiazolin 4 one, etc. Suitable fluorescent or fluorogenic labels include rhodamine 110; rhodol; coumarin or a fluorescein compound. Derivatives of rhodamine 110, rhodol, or fluorescein compounds that have a 4′ or 5′ protected carbon may likewise be employed. Preferred examples of such compounds include 4′(5′)thiofluorescein, 4′(5′)-aminofluorescein, 4′(5′)-carboxyfluorescein, 4′(5′)-chlorofluorescein, 4′(5′)-methylfluorescein, 4′(5′)-sulfofluorescein, 4′(5′)-aminorhodol, 4′(5′)-carboxyrhodol, 4′(5′)-chlororhodol, 4′(5′)-methylrhodol, 4′(5′)-sulforhodol; 4′(5′)-aminorhodamine 110, 4′(5′)-carboxyrhodamine 110, 4′(5′)-chlororhodamine 110, 4′(5′)-methylrhodamine 110, 4′(5′)-sulforhodamine 110 and 4′(5′)thiorhodamine 110. “4′(5′)” means that at the 4 or 5′ position the hydrogen atom on the carbon atom is substituted with a specific organic group or groups as previously listed. A 7-Amino, or sulfonated coumarin derivative may likewise be employed. Any of a variety of cyanine dyes, such as those disclosed in U.S. Pat. Nos. 2,734,900, 6,002,003, or 6,110,630 may likewise be employed. The use of enzymes (especially alkaline phosphatase, β-galactosidase, horse radish peroxidase, or urease) as detectable label (i.e., an enzyme immunoassay or EIA) is preferred.

In one embodiment of the invention, the ligand and ligand-binding molecule are selected so that the target cell is essentially irreversibly immobilized to the support. Alternatively, a ligand and ligand-binding molecule are employed that can be dissociated from one another by binding competitors (e.g., pharmacological agents, binding inhibitors, hormone analogs, mimetics, etc.).

The invention is particularly amenable for use in the A² platform and the IC100 platform (Beckman Coulter, Inc.) (U.S. Pat. No.5,648,213 (Reddy, M. P)) in conjunction with universal linker technology. Such technology uses the hybridization of carefully screened oligonucleotides to selectively immobilize target entities to specific areas on a DNA microarray under physiological conditions.

The use of oligonucleotide linker technology to generate microarrays of relatively labile species, such as antibodies, has a number of advantages, including:
1. Use of a single set of printing conditions for all immobilized species.
2. Long term stability of the microarray-oligonucleotides are very stable molecules compared to proteins.
3. The Universal Linker technology makes these microarrays addressable, so a single type of printed microarray may be used to generate an extremely wide variety of functional microarrays.
4. Universal Linker technology may be used to generate microarrays of labile species that cannot be subjected to common encountered printing conditions, such as drying.
5. Universal Linker technology may be used to generate a microarray that contains a variety of different molecules, for example monoclonal antibodies, synthetic peptides, and analogs of drugs that all interact with cell surface markers.
6. Universal Linker technology provides a spacer group between the ligand and the support, which may be tailored to meet desired criteria such as length, hydrophobicity, charge, etc.
7. Universal Linker technology provides selective elution of immobilized cells by convenient, low toxicity means, such as competition with free complementary oligonucleotide.

The A² System (Beckman Coulter, Inc.) is a multiplexed immunoassay technology that can measure multiple proteins within a single well, and which is particularly amenable to use in accordance with the principles of the present invention. The A² system employs an A² Plate, which comprises an array within an array and can measure multiple analytes per well through the use of a set of oligonucleotides of different sequence that are printed onto the surface of the well. The A² Plate may be used as the microarray support of the present invention, and the printed oligonucleotide may be employed as the substrate-immobilized oligonucleotide of the present invention. The bioconjugate molecules of the present invention can be formulated to possess an oligonucleotide component whose sequence is complementary to the sequence of the printed oligonucleotide. The bioconjugate molecules can then be added to the wells of the plate and will hybridize to the immobilized printed oligonucleotides of the plate, thus creating a desired microarray. Since multiple oligonucleotide species can be printed to the plate, multiple bioconjugate species can be immobilized to the same A² plate.

This technology can also be used to generate microarrays of intact viruses, which are of considerable interest in the pharmaceutical industry for vaccine research.

Another application of this invention is the generation of mixed-mode microarrays that combine receptor-based and cell-based assays. The universal linker approach permits targeting of a variety of molecules to a conventional oligonucleotide microarray at high fidelity under essentially physiological conditions. It is therefore possible to use in situ hybridization to generate microarrays that contain sites specifically designed for capture of live cells and for immunoassays.

### D. Microarray Format

The present invention permits users to independently select desired target cells, ligands and ligand binding-molecules. As a consequence, the present invention provides a very broad range of different procedures and formats. Desired assay formats can be produced by adapting teachings related to antibody and protein microarrays (e.g., Panicker, R. C. et al., “Recent Advances In Peptide-Based Microarray Technologies,” Comb. Chem. High Throughput Screen. September 2004;7(6):547-556; Pavlickova, P. et al., “Advances In Recombinant Antibody Microarrays,” Clin. Chim. Acta. May 2004;343(1-2):17-35); Chen, G. Y. et al., “Array-Based Technologies And Their Applications In Proteomics,” Curr. Top. Med. Chem. 2003 ;3(6):705-724; Nielsen, U. B. et al., “Multiplexed Sandwich Assays In Microarray Format,” J. Immunol. Methods July 2004;290(1-2):107-120; Bailey, S. N. et al., “Microarrays Of Small Molecules Embedded In Biodegradable Polymers For Use In Mammalian Cell-Based Screens,” Proc. Natl. Acad. Sci. U.S.A. Nov. 16, 2004;101(46):16144-9. Epub Nov. 16, 2004; U.S. Patent Applns. Publn. Nos. US2004/0033546 (Wang, D.), US2003/0153013 (Huang, R. P.); US2003/0108972 (Zweig, S. E. et al.); US2003/0108949 (Bao, G. et al.); 2002/0164656 (Hoeffler, J. P. et al.); PCT Publn. WO99/40434 (Hoeffler, J. P. et al.); PCT Publn. No. WO2004/076678 (Green, L.); PCT Publn. No. WO2004/005477 (Charych, D. et al.); PCT Publn. No. WO02/073180 (Huang, R. P.); PCT Publn. No. WO02/39120 (George, S. T. et al.); PCT Publn. No. WO02/12893 (Cardone, M. H. et al.); PCT Publn. No. WO00/63701 (Brown, P. et al.); PCT Publn. No. WO03/003014 (Pearce, C. D. J. et al.); PCT Publn. No. WO02/083918 (Wang, D.); PCT Publn. No. WO01/36585 (Anderson, N. L.); U.S. Pat. No. 5,648,213 (Reddy, M. P. et al.); Jackson, A. M. et al., “Cell-Free Protein Synthesis For Proteomics,” Brief Funct. Genomic Proteomic. February 2004;2(4):308-319); Oleinikov, A. V. et al., “Self-Assembling Protein Arrays Using Electronic Semiconductor Microchips And In Vitro Translation,” J. Proteome Res. May-June 2004;2(3):313-319; Weng, S. et al., “Generating Addressable Protein Microarrays With Profusion Covalent mRNA-Protein Fusion Technology,” Proteomics. January 2002;2(1):48-57; etc.). In one embodiment, the microarrays of the present invention can be employed in direct assays to determine the presence or extent of any cellular binding to a microarray. By way of illustration, such a direct assay could involve forming a microarray in which a ligand-binding molecule-oligonucleotide bioconjugate is immobilized to a support through oligonucleotide-oligonucleotide hybridization with a support-immobilized oligonucleotide (FIG. 2). The ligand-binding molecule of the bioconjugate is selected for its capacity to bind to a ligand on the surface of the desired target cell. The presence or extent of any cellular binding to the microarray may be determined visually (e.g., a cell count, etc.) or more preferably through the use of a detectably labeled reagent target cell. The presence or extent of detectable label found to be immobilized to the microarray is indicative of the presence or extent of target cell immobilized to the microarray. In an alternative embodiment, the microarrays of the present invention can be employed in indirect assays to determine the presence or extent of any cellular binding to a microarray. In such an embodiment, a detectably labeled reagent is employed that is capable of specific binding to the bioconjugate molecule provided that the antibody component of the bioconjugate is not bound to a target cell. The presence or extent of binding of such reagent to the support is inversely proportional to the presence or extent of target cells in the sample being evaluated. Blood typing and cell typing can be accomplished using the microarrays of the present invention.

The methods and compositions of the present invention can also be employed to assay internal components of a cell, especially internal components whose presence or expression is characteristic of a morphological state (such as an apoptotic state, an inflammatory state, etc.) or a disease state (such as a tumorigenic state). In one embodiment of such use, desired target cells are incubated (before or after immobilization to a microarray of the invention, with an assay compound having the ability to pass through the cell's membrane. For example, cell viability can be assessed through the use of stains, such as trypan blue, that indicate cell viability. Internal cellular components, and their expression can be assayed, for example, by providing the cells with an assay compound having (i) a leaving group selected so that it may be cleaved by an enzyme to be analyzed and (ii) a fluorogenic indicator group selected for its ability to have a non-fluorescent first state when joined to the leaving group, and a fluorescent second state excitable at a convenient wavelength (e.g., a wavelength above 450 nm) when the leaving group is cleaved from the indicator group by the enzyme (U.S. Pat. No.5,698,411 (Lucas, et al.); U.S. Pat. 5,976,822 (Landrum et al.). Exemplary assay compounds possess an unblocked leaving group selected for cleavage by an enzyme to be analyzed (such as a cysteine protease (especially a caspase enzyme or a granzyme of cysteine proteases), dipeptyl peptidase and calpain), and a fluorogenic indicator group selected for its ability to have a non-fluorescent first state when joined to the leaving group, and a fluorescent second state excitable at a wavelength when the unblocked leaving group is cleaved from the indicator group by the enzyme. Various indicator groups are disclosed (4′(5′)aminorhodamine 110, 4′(5′)carboxyrhodamine 110, 4′(5′)chlororhodamine 110, 4′(5′)methylrhodamine 110, 4′(5′)sulforhodamine 110, 4′(5′)aminorhodol, 4′(5′)carboxyrhodol, 4′(5′)chlororhodol, 4′(5′)methylrhodol, 4′(5′)sulforhodol, 4′(5′)aminofluorescein, 4′(5′)carboxyfluorescein, 4′(5′)chlorofluorescein, 4′(5′)methylfluorescein, and 4′(5′)sulfofluorescein). Agents (such as glycerol, dimethyl sulfoxide (DMSO), trehalose, glutamate, betaine, ethylene glycol, threitol, ribose, trimethylamine N-oxide, etc.) that promote increased uptake of molecules into metabolically active cells may be provided (U.S. Patent Appln. Publn. No. 20030077569 (Clausell et al.). A wide variety of molecules can be monitored in this matter (e.g. 5′ nucleotidases, acetylcholinesterases, acid phosphatases, acidic esterases (e.g., acidic esterase I, acidic esterase II, acidic non-specific esterase, etc.), adenosine deaminases (e.g., adenosine monophosphate deaminase, etc.) alkaline phosphatases, aminopeptidases (e.g., aminopeptidase A, aminopeptidase B, aminopeptidase M, Aminopeptidase N, etc.) angiotensin converting enzyme, cathepsins (e.g., cathepsin B, cathepsin B1, cathepsin C, cathepsin D, cathepsin H, cathepsin L, etc.), cholinesterases, chymotrypsins, collagenases, cytosine deaminases, DPP I, DPP II, DPP IV, elastases, endopesidases (e.g., endopeptidase I, endopeptidase II, membrane associated endopeptidase I, a membrane associated endopeptidase II, a neutral endopeptidase,etc.) ester proteinases, galactopyranosidases, glucoronidases, glutathione, glycopyranosidases, guanine deaminases, HIV Proteases, interleukin-1β converting enzymes (“ICE,” also known as “caspases”), lipases, neutral esterases (e.g., neutral esterase I, neutral esterase II, neutral non-specific esterase, etc.), nucleosidases, pancreatins, phospholipases (e.g., phospholipase A, phospholipase C, phospholipase D, etc.), plasmins, phospahatases (e.g., serine phosphatase, tartrate resistant phosphatase, threonine phosphatase, tyrosine phosphatase, etc.), thymidine deaminases, tripeptidyl peptidases, trypsins, urokinases, γ-Glutamyl Transferases, etc. The assaying of such components can be used to determine the presence or absence of an apoptotic state, and can aid in the diagnosis of cancer (e.g., cervical cancer), diagnosis of viral replication in HIV patients, diagnosis of HIV infected blood in blood supply, diagnosis of TB infected HIV patients, diagnosis of improved blood differential, differential diagnosis of viral from bacterial infections, differential diagnosis of Lupus from rheumatoid arthritis, differential diagnosis between rheumatoid arthritis from osteoarthritis, diagnosis of vasculitis, diagnosis of cardiovascular disease, monitoring of chemotherapeutic efficacy, diagnosis of Hodgkins Disease, confirmation of gene implantation and diagnosis of transplant rejection. The cell-based microarrays of the present invention thus can be used to assay inflammation by, for example, identifying indicators of inflammation.

The cell-based microassays of the present invention can also be used to assay G protein-coupled receptors (“GPCRs”) (see, U.S. Pat. No. 6,770,449 (Barak et al.), U.S. Pat. No. 5,891,646 (Barak et al.), U.S. Pat. No. 6,110,693 (Barak et al.), U.S. Pat. No. 6,528,271 (Bohn et al.), PCT Appln. Publn. No. WO9855635 (Barak et al.), WO0020590 (Tang et al.). GRCRs comprise a large superfamily of proteins, which includes: the A2a adenosine receptor; the A2b adenosine receptor; the β1-adrenergic receptor; the β2-adrenergic receptor; the CRF1 corticotropin releasing factor receptor; the D1 dopamine receptor; the D5 dopamine receptor; the FSH follicle-stimulating hormone receptor; the Glucagon receptor; the LH luteinizing hormone receptor; the PTH1 parathyroid hormone receptor; the E2 prostaglandin receptor; the E4 prostaglandin receptor; the Secretin receptor; the VIP1 vasoactive intestinal peptide receptor; the V2 vasopressin receptor; the; the α2a-adrenergic receptor; the α2b-adrenergic receptor; the α2c-adrenergic receptor; the A1 adenosine receptor; the A3 adenosine receptor; the Apelin receptor; the C5a anaphylatoxin receptor; the CCR5 receptor; the CXCR1 receptor; the CXCR2 receptor; the CXCR4 receptor; the D2 dopamine receptor; the D3 dopamine receptor; the D4 dopamine receptor; the Edg1 receptor; the Edg2 receptor; the Edg3 receptor; the Edg5 receptor; the 5HT1A hydroxytryptamine receptor; the δ-opioid receptor; the μ-opioid receptor; the MCH1 melanin conc. hormone receptor; the M2Ach muscarinic acetylcholine receptor; the E3 prostaglandin receptor; theormyl peptide receptor; the Neuropeptide FF receptor; the ha 1b-adrenergic receptor; the AT1A angiotensin II receptor; the CCK-A cholecystokinin receptor; the CCK-B cholecystokinin receptor; the Cytomegalovirus US28 receptor; the ETA endothelin receptor; the GnRH (type2) gonadotropin releasing hormone receptor; the 5HT2A hydroxytryptamine receptor; the 5HT2C hydroxytryptamine receptor; the m1ACh muscarinic acetylcholine receptor; the mGluR1 metabotropic glutamate receptor; the NK1 neurokinin receptor; the NK3 neurokinin receptor; the NT1 neurotensin receptor; the Orexin-1 receptor; the Oxytocin receptor; the PAR2 proteinase-activated receptor; the Platelet-activating factor receptor; the TRHR-1 thyrotropin releasing hormone receptor; the TRHR-2 thyrotropin releasing hormone receptor; and the Somatostatin receptor. Individual GPCR types activate a particular signal transduction pathway; at least ten different signal transduction pathways are known to be activated via GPCRs (the β 2-adrenergic receptor (βAR) is a prototype mammalian GPCR. In response to agonist binding, βAR receptors activate a G protein (Gₛ) which in turn stimulates adenylate cyclase and cyclic adenosine monophosphate production in the cell. Many available therapeutic drugs in use today target GPCRs, as they mediate vital physiological responses, including vasodilation, heart rate, bronchodilation, endocrine secretion, and gut peristalsis (Lefkowitz et al., Ann. Rev. Biochem. 52:159 (1983). For example, ligands to βARs are used in the treatment of anaphylaxis, shock, hypertension, hypotension, asthma and other conditions.

The cell-based microarrays of the present invention may be used to facilitate drug discovery or the testing of consumer products (e.g., cosmetics, soaps, perfumes, etc.), foodstuffs, biohazard pathogens, etc.

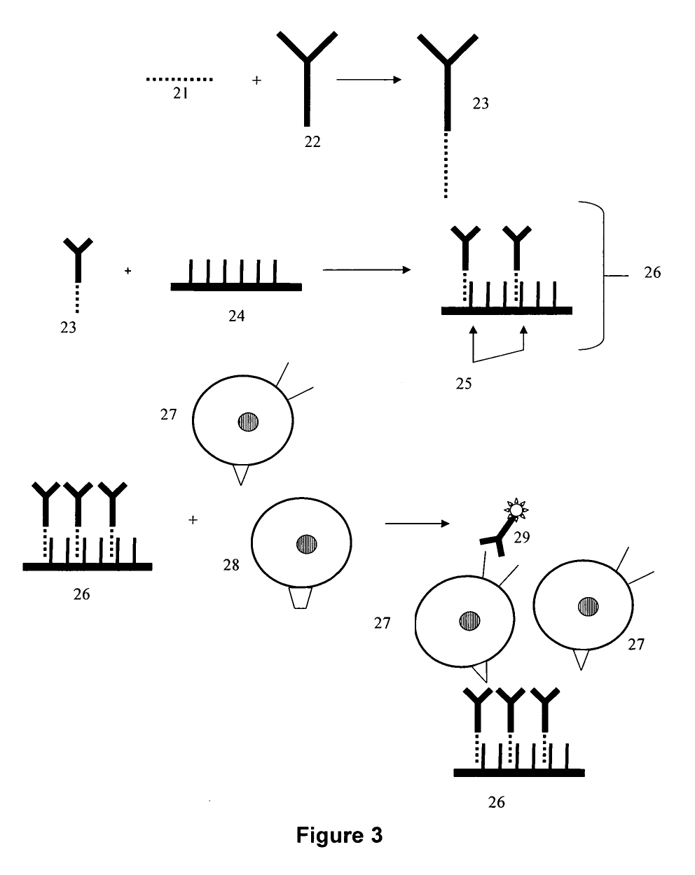

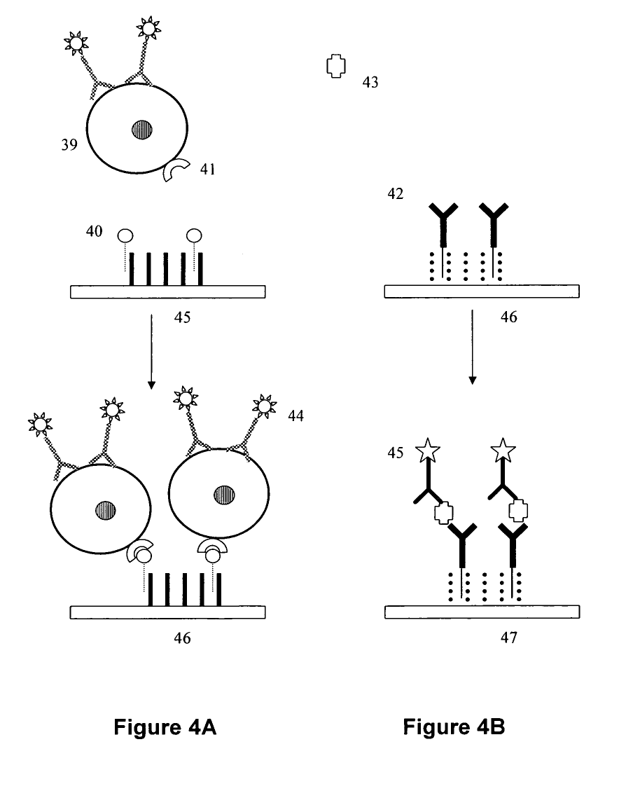

The microarrays of the present invention can be employed in sandwich assays to determine the presence or extent of any cellular binding to a microarray (FIG. 3, FIG. 4A, FIG. 4B,). Such sandwich assays may be formatted so as to be either homogeneous or heterogeneous in nature. They may be competitive or non-competitive. U.S. Pat. Nos. 5,976,822; 5,876,935; 5,851,778; 5,811,526; 5,747,352; 5,698,411; 5,691,147; 5,679,525; 5,633,141; 5,627,080; 5,563,036; 4,016,043; and 3,791,932 illustrate several different assay formats and applications that may be adapted to employ the microarrays of the present invention.

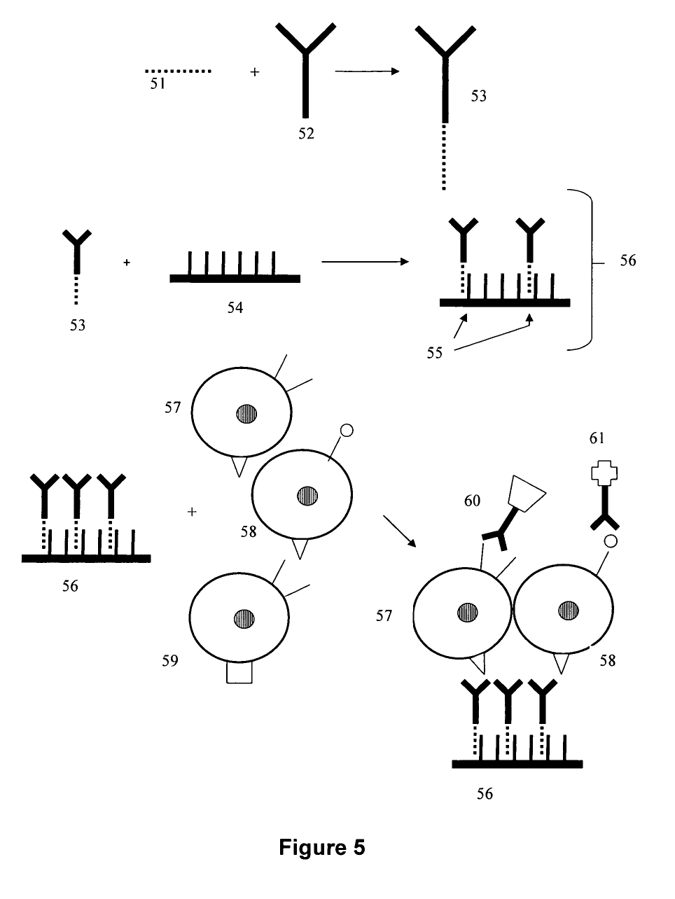

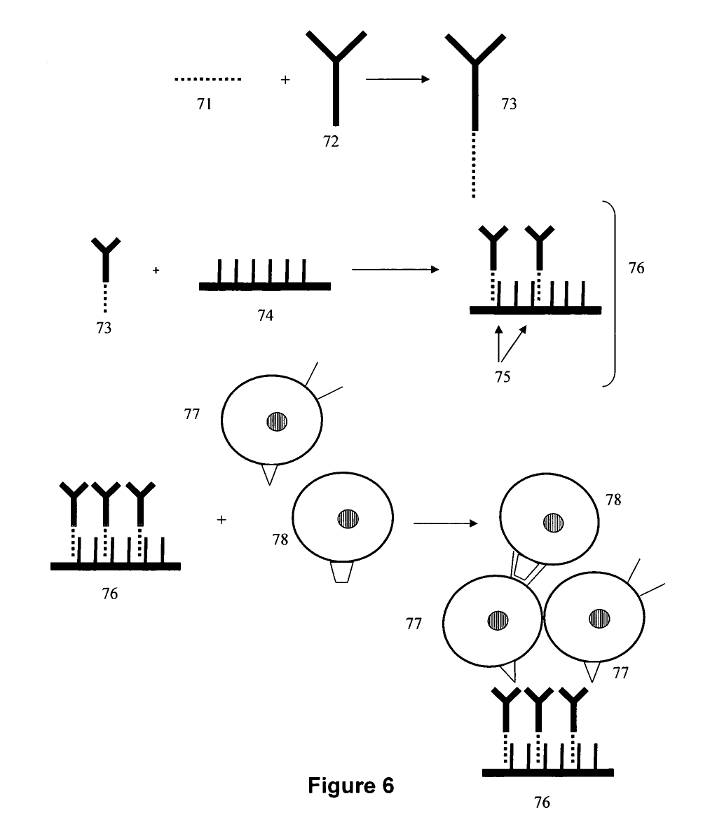

In one embodiment of such an assay, the immobilization of target cells may be determined using a detectably labeled reagent capable of binding to the target cell (FIG. 5). The presence or extent of the detectable label of such reagent found to be immobilized to the microarray is indicative of the presence or extent of target cell in the sample being evaluated. In a second embodiment, a cell (preferably labeled or capable of binding to a detectably labeled reagent) containing a ligand that binds to a ligand of the immobilized target cell may be used to enable the detection of the target cell (FIG. 6). In such an embodiment, the labeled cell may be labeled using an antibody or other molecule, or it may be itself labeled.

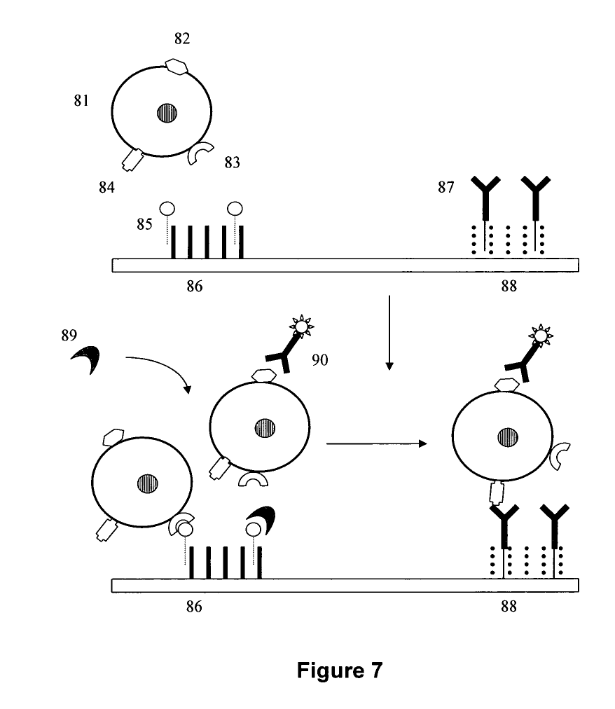

As a further example, the present invention permits the formation of homogenous immunochromatographic assay formats. In a first preferred immunochromatographic assay format, the microarray will contain at least two contacting, but spatially distinct, regions. The first such region will contain an immobilized bioconjugate whose ligand-binding molecule component is bound to a detectably labeled target cell (FIG. 7). As in the examples described above, the detectable label may comprise a direct labeling of the cell, or it may comprise an indirect labeling (e.g., the use of a labeled ligand-binding molecule that is bound to, or capable of binding to, the target cell). The second such region will contain a second immobilized molecule (e.g., an antibody, a bioconjugate, etc.) capable of binding to the target cell. Most preferably, the immobilized molecule of the second region will recognize a different ligand from that recognized by the bioconjugate employed in the first region. The detection of label immobilized to the second region is indicative of the presence of an agent capable of competing with the binding of the target cell to the ligand-binding molecule of the bioconjugate employed in the first region of the microarray. Such a microarray may be employed to detect pharmacological agents, hormones, blood factors, etc.

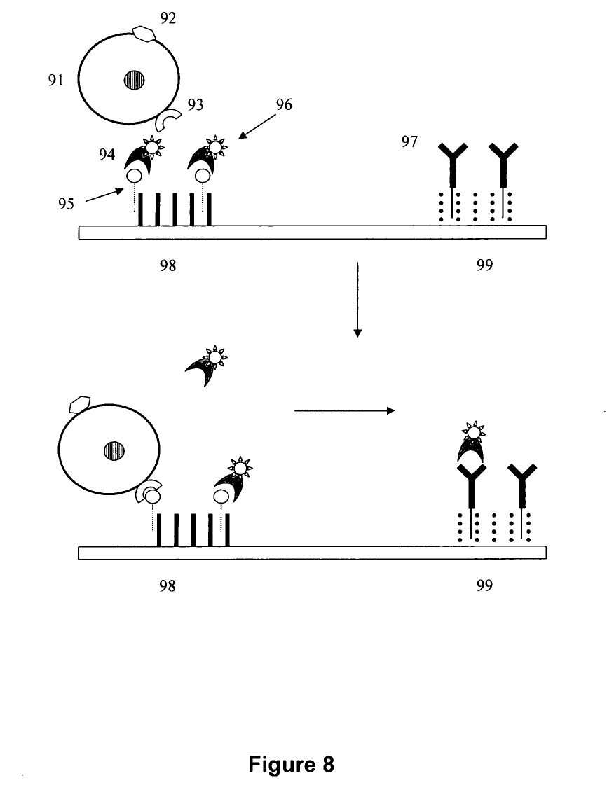

In a second preferred immunochromatographic assay format, the microarray will again contain at least two contacting, but spatially distinct, regions. The first such region will contain an immobilized bioconjugate whose ligand-binding molecule component is bound to a detectably labeled molecule (e.g., a candidate pharmacological agent, a hormone, a soluble receptor ligand, etc.) (FIG. 8). The second such region will contain a second immobilized molecule (e.g., an antibody, a bioconjugate, etc.) capable of binding to the detectably labeled molecule. The detection of label immobilized to the second region is indicative of the presence of cells having ligands that are capable of competing with the binding of the detectably labeled molecule to the ligand-binding molecule of the bioconjugate employed in the first region of the microarray. Such a microarray may be employed to detect the presence or extent of desired types of target cells in a biological sample.

In one embodiment, such microarrays will comprise a hollow casing constructed of, for example, a plastic material, etc., in which the first region will communicate indirectly with the interior of the casing via, for example, a multilayer filter system that is accessible from the device (e.g., by protruding therefrom or by being incompletely covered by the device), such that a test sample can be applied directly to the filter system and will permeate therefrom into the first region. In such a device, the permeation of fluid containing the target material (e.g., target cell, biomolecule, etc.) will cause the target material to compete for binding with any detectably labeled molecule or target cell that has been immobilized to the bioconjugate of the first region, thereby releasing such detectably labeled entities so that they may permeate into the second region of the microarray.

Detection of labeled material in the second region of the microarray thus indicates that the target material is present in the sample being evaluated. The assay can be made quantitative by measuring the quantity of labeled material that becomes bound to the second region of the microarray.

While the microarrays of the present invention have been described above in terms of their ability to be used in assays of cell binding and of cellular ligands, it will be appreciated that the invention permits the purification of cell types and cellular sub-types, and thus also permits the isolation of a selected population of cells, or a selected sub-population of cells. Such isolated cells can then be used to conduct cell-based assays of their metabolic activity. For example, a sample, which may be unpurified, partially purified or highly purified, may be placed in contact with a microarray containing immobilized anti-ligand bioconjugate molecules, so as to cause desired target cells to become immobilized to the support. Undesired cells and other materials may be removed by washing or other methods. This aspect of the present invention provides the advantage of being able to readily collect, and hence concentrate, desired target cells of a sample. Additionally, by employing microarray regions or zones that contain different immobilized bioconjugates, it is possible to isolate sub-populations of the desired target cell and distinguish their respective activities from one another.

The resulting microarray can then be used to assay any of a variety of cellular processes. Indeed, any of a wide variety of enzymes, proteins, etc. may be analyzed in accordance with the principles of such aspect of the present invention. In particular, the activity or presence of cellular enzymes, including proteases, glycosidases, glucosidases, carbohydrases, phosphodiesterases, phosphatases, sulfatases, thioesterases, pyrophosphatases, lipases, esterases, nucleotidases and nucleosidases may be analyzed. As used herein, the term “carbohydrase” includes any enzyme that has the ability to hydrolyze a carbohydrate. Enzymes which do not recognize and cleave a leaving group, such as dehydrogenases and kinases, are not preferred for assays according to the invention. The enzymes to be measured can be those that are present in various cell preparations, enzymes found in cytosols, cell surface enzymes, cytoplasmic enzymes and cell nucleus (nuclear) enzymes. However, the principles of this aspect of the present invention are particularly useful for detecting or analyzing intracellular enzymes in living cells. Additional enzymes whose activity or presence may be measured in accordance with the present invention include: 5′ nucleotidase, acetylcholinesterase, acid phosphatase, acidic esterase, acidic esterase I, acidic esterase II, acidic non-specific esterase, adenosine deaminase, adenosine monophosphate deaminase, alkaline phosphatase, aminopeptidase A, aminopeptidase B, aminopeptidase M, Aminopeptidase N, angiotensin converting enzyme, caspase (including caspases 1, 3 6, 8, or 9), cathepsin B, cathepsin B1, cathepsin C, cathepsin D, cathepsin H, cathepsin L, cholinesterase, cholinesterase, chymotrypsin, collagenase, cytosine deaminase, DPP I, DPP II, DPP IV, elastase, endopeptidase I, endopeptidase II, ester proteinase, galactopyranosidase, glucoronidase, glutathione, glycopyranossidase, guanine deaminase, HIV Protease, lipase, membrane associated endopeptidase I, membrane associated endopeptidase II, neutral endopeptidase, neutral esterase, neutral esterase I, neutral esterase II, neutral non-specific esterase, nucleosidase, pancreatin, phospholipase A, phospholipase C, phospholipase D, plasmin, serine phosphatase, tartrate resistant phosphatase, tartrate resistant phosphatase, threonine phosphatase, thymidine deaminase, tripeptidyl peptidase, trypsin, tyrosine phosphatase, urokinase, v-thrompsin, and γ-GT.

The microarrays of the present invention are also capable of facilitating in situ biochemical assays. In one embodiment, such assays comprise an in vitro nucleic acid amplification process such as, for example, the Polymerase Chain Reaction (U.S. Pat. No. 4,582,788 (Erlich et al.); U.S. Pat. No. 4,683,194 (Saiki et al.); U.S. Pat. No. 4,683,202 (Mullis et al.)), the Ligase Chain Reaction (U.S. Pat. No. 5,427,930 (Birkenmeyer et al.); U.S. Pat. No. 5,516,663 (Backman et al.)), End-Run Amplification (U.S. Pat. No. 6,180,338 (Adams)), Rolling Circle Amplification (U.S. Pat. No. 5,354,668 (Auerbach), U.S. Pat. No. 5,854,033 (Lizardi et al.); U.S. Pat. No. 6,740,745 (Auerbach); U.S. Pat. No. 5,876,924 (Zhang et al.)), Strand Displacement Amplification (U.S. Pat. No. 5,270,184 (Walker et al.)), NASBA (U.S. Pat. No. 5,409,818 (Davey et al.)), etc. In one embodiment, such analyses may be used to facilitate the genotyping, haplotyping, diagnosis and/or detection of mutations, alleles, or polymorphisms (especially single nucleotide polymorphisms (SNPs).

In a further embodiment, the microarrays of the present invention may be used to facilitate “polony” (“polymerase-colony) analysis of the immobilized cells (or colonies or clusters of immobilized cells). In such analyses, the microarray matrix retards the diffusion of the amplified nucleic acid molecules, thereby permitting the amplification products to remain localized near their respective templates (Mitra, R. D. et al. “In Situ Localized Amplification And Contact Replication Of Many Individual DNA Molecules,” Nucleic Acids Res. 1999 27(24):e34; pp. 1-6; Mitra, R. D. et al. “Digital Genotyping and Haplotyping with Polymerase Colonies,” Proc Natl Acad Sci USA. 2003 100(10):5926-5931; Merritt, J. et al. “Parallel Competition Analysis Of Saccharomyces Cerevisiae Strains Differing By A Single Base Using Polymerase Colonies,” Nucleic Acids Res. 2003 31(15):e84; Mitra, R. D. et al. “Fluorescent in situ Sequencing on Polymerase Colonies,” Analyt. Biochem. 2003 320:55-65; Zhu, J. et al. “Single Molecule Profiling of Alternative Pre-mRNA Splicing,” Science 2003 301(5634):836-838; Aach, J et al. “Mathematical Models Of Diffusion-Constrained Polymerase Chain Reactions: Basis Of High-Throughput Nucleic Acid Assays And Simple Self-Organizing Systems,” J. Theoret. Biol. 2004 228(l):31-46; Constans, A “Beyond Sanger: Toward the $1000 Genonme,” The Scientist 2003 17:36; Butz, J. et al. “Characterization Of Mutations And Loss Of Heterozygosity Of p 53 and K-ras2 In Pancreatic Cancer Cell Lines By Immobilized Polymerase Chain Reaction,” BMC Biotechnol. 2003 3(1):11; Dressman, D. et al. “Transforming Single DNA Molecules Into Fluorescent Magnetic Particles For Detection And Enumeration Of Genetic Variations,” Proc Natl Acad Sci U S A. 2003 100(15):8817-8822; Mikkilineni, V. et al. “Digital Quantitative Measurements Of Gene Expression,” Biotechnology and Bioengineering 2004 86(2): 117-124; Shendure, J. et al. “Advanced Sequencing Technologies: Methods and Goals,” Nature Reviews of Genetics 2004 5(5):335-344; Shendure, J. et al. “Accurate Multiplex Polony Sequencing of an Evolved Bacterial Genome,” Science 2005 (epub)).

Thus, the invention permits in situ nucleic acid amplification to be detected from within immobilized cells possessing a desired target nucleic acid molecule. Such a cell-based “polony” procedure is particularly facilitated by the use of latex bead or polypropylene supports.

The present invention is also particularly amendable to the construction and use of arrays capable of determining the presence, nature, or absence of blood typing markers (A, B, Rₕ, M, N, etc.) on the surfaces of erythrocytes. For such an embodiment, an array may be formed using oligonucleotides capable of hybridizing to complementary oligonucleotides that are conjugated to, for example, antibodies that are able to immunologically recognize and bind to blood typing markers. Incubation of the array with a blood sample followed by washing away unbound material would permit a rapid and detailed identification of blood type, including rare forms, from a single test. Likewise, the microarrays of the present invention permit cell typing (e.g., distinguishing between CD4⁺ and CD4⁻ lymphocytes, etc.).

The present invention is amenable to conducting such assays in accordance with the principles of Lucas et al., (U.S. Pat. No. 5,698,411) and Landrum et al., (U.S. Pat. No. 5,976,822, and, more preferably, the principles of Clausell et al., (WO 03034025) in which one or more agents that cause the increased uptake of analytes and/or substrates are included in the assay. Suitable detection methods and reagents are disclosed by Clausell et al., (WO 03034025). Without in any way intending to define the mechanism of action of the agents of the present invention, such agents include those that induce hyperosmotic shock, and have the general characteristic of being able to help stabilize or fold proteins and/or assist organisms that experience osmotic shock or need to stabilize themselves from osmotic shock. Such agents include glycerol, dimethyl sulfoxide (DMSO), trehalose, glutamate, betaine, ethylene glycol, threitol, ribose, trimethylamine N-oxide, etc. The use of such agents has been found to result in increased uptake and/or transport of substrates and analytes. Such increased uptake and/or transport thus to enhance the sensitivity of substrate or analyte detection, and result in improved assays. The invention further concerns the embodiments of such methods wherein the uptake-enhancing agent is selected from the group consisting of glycerol (especially wherein the glycerol concentration is between about 5% and about 60% (v/v), or between about 20% and about 60% (v/v) or between about 25% and about 40% (v/v)), dimethyl sulfoxide (DMSO) (especially wherein the DMSO concentration is between about 5% and about 60% (v/v), or between about 20% and about 60% (v/v)), trehalose (especially wherein the trehalose concentration is between about 0.1 M and about 1.5 M), glutamate (especially wherein the glutamate concentration is between about 0.25 M and about 2.0 M, or between about I M and about 2 M), betaine (especially wherein the betaine concentration is about 0.3 M or greater), ethylene glycol (especially wherein the ethylene glycol concentration is between about 2 M and about 7 M), threitol (especially wherein the threitol concentration is between about 1 M and about 5 M), ribose (especially wherein the ribose concentration is between about 0.4 M and about 4 M), and trimethylamine N-oxide (especially wherein the trimethylamine N-oxide concentration is between about 0.4 M and about 4 M). The provision of agents that cause increased uptake of analytes and/or substrates may destabilize the duplex stability of DNA duplex and thereby lead to a loss of array sensitivity. In those circumstances in which such destabilization occurs to an unacceptable degree, improved stability can be achieved by lowering the array temperature, increasing the salt concentration, or employing agents (e.g., glycerol, etc.) that are less disruptive of duplex stability (Bonner, G. et al. (2000) Biotech Bioeng. 68(3): 339-344).

All publications and patent applications mentioned in this specification are herein incorporated by reference to the same extent as if each individual publication or patent application had been specifically and individually indicated to be incorporated by reference. The discussion of the background to the invention herein is included to explain the context of the invention. Such explanation is not an admission that any of the material referred to was published, known, or part of the prior art or common general knowledge anywhere in the world as of the priority date of any of the aspects listed above.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A cell-based microarray, comprising:
(A) a target cell having a surface ligand;
(B) one or more species of bioconjugate molecules, each such molecules comprising a ligand-binding molecule portion conjugated to an oligonucleotide molecule portion, and each such species having a different ligand-binding portion, and
(C) a support having immobilized thereto one or more species of oligonucleotide molecules, each such species having different a different nucleotide sequence,
wherein an oligonucleotide portion of a bioconjugate molecule and a support-immobilized oligonucleotide are hybridized to one another, and wherein said ligand-binding molecule of said hybridized bioconjugate molecule is bound to said surface ligand of said target cell, thereby immobilizing said target cell to said support.

2. The cell-based microarray of claim 1, wherein said microarray comprises a plurality of different species of target cells each such species bound to a different species of bioconjugate molecule, wherein said different species of bioconjugate molecule are hybridized to an ordered array of oligonucleotide species immobilized to said support.

3. The cell-based microarray of claim 1, wherein said solid support is glass, paper, optical fiber, or plastic.

4. The cell-based microarray of claim 1, wherein said target cell is a mammalian cell, a reptilian cell, an avian cell, a fish cell, a fungal cell, a plant cell, a yeast cell, a bacterial cell, or viral particle.

5. The cell-based microarray of claim 1, wherein said surface ligand is an antigenic surface protein, a receptor, a transmembranous enzyme, that is naturally present on the surface of normal target cells.

6. The cell-based microarray of claim 1, wherein the presence of said surface ligand is associated with a disease state.

7. The cell-based microarray of claim 1, wherein the ligand binding molecule is an immunoglobulin, a hormone, an immunomodulator, a cytokine, a chemokine, a pharmacological agent or a substrate or inhibitor of a transmembranous enzyme.

8. The cell-based microarray of claim 1, wherein a molecule of a species of one of said bioconjugate molecules is formed by a method that comprises the steps of:
(A) contacting an oligonucleotide having an amino group with a heterofunctional linker, wherein said linker has a first group reactive with said amino group and a second group reactive with a thiol group, said contacting being under conditions sufficient to permit said first group of said heterofunctional linker to become bonded to said amino group of said oligonucleotide, thereby forming an oligonucleotide-heterofunctional linker conjugate; and
(B) contacting said oligonucleotide-heterofunctional linker conjugate (A) with a protein having a thiol group reactive with said second group of said heterofunctional linker; said contacting being under conditions sufficient to permit said thiol group of said protein to become bonded to said second group of said heterofunctional linker of said oligonucleotide-heterofunctional linker conjugate, to thereby form a molecule of a species of said bioconjugate molecules.

9. The cell-based microarray of claim 8, wherein said first group of said heterofunctional linker is an NHS group, and said second group of said heterofunctional linker is a maleimide group.

10. The cell-based microarray of claim 8, wherein said heterofunctional linker is selected from the group consisting of Sulfo-SMCC; Sulfo-EMCS; Sulfo-GMBS; Sulfo-KMUS; Sulfo-MBS; Sulfo-SIAB; Sulfo-SMPB; Sulfo-LC-SMPT; SVSB; SIACX; SIA, SIAXX; and NPIA.

11. The cell-based microarray of claim 1, wherein said microarray assays the viability of said target cell.

12. The cell-based microarray of claim 1, wherein said microarray assays blood type.

13. The cell-based microarray of claim 1, wherein said microarray assays cell type.

14. The cell-based microarray of claim 1, wherein said microarray assays the presence or expression of an internal component of said target cell.

15. The cell-based microarray of claim 14, wherein said presence or expression of said internal component is characteristic of an apoptotic state or a disease state.

16. The cell-based microarray of claim 1, wherein said microarray assays for the presence of a nucleic acid molecule produced within said immobilized cell.

17. A method for determining whether a population of cells contains a target cell that possesses a desired surface ligand, said method comprising the steps:
(A) incubating said population of cells in the presence of:
(1) one or more species of bioconjugate molecules, each such species comprising a ligand-binding molecule portion conjugated to an oligonucleotide molecule portion, wherein at least one of said species of bioconjugate molecules comprises a ligand-binding molecule portion capable of binding to said desired surface ligand; and
(2) a support having immobilized thereto one or more species of oligonucleotide molecules, each such species having a different nucleotide sequence, and at least one species being capable of hybridizing to the nucleotide sequence of the oligonucleotide of said bioconjugate molecule,
wherein said incubation is conducted under conditions sufficient to permit:
(a) hybridization between complementary nucleotide sequences of said bioconjugate and said support-immobilized oligonucleotide; and
(b) binding between a ligand-binding molecule of said bioconjugate molecules and said desired surface ligand of said target cell to thereby immobilize said target cell to said support; and
(B) determining whether any cell of said population possess said surface ligand by detecting the immobilization of cells to said surface, wherein said immobilization is achieved through the binding of said target cell's surface ligand to the ligand-binding molecule of a bioconjugate whose oligonucleotide portion has hybridized to a support-immobilized oligonucleotide.

18. The method of claim 17, wherein said microarray comprises a plurality of different species of target cells each such species bound to a different species of bioconjugate molecule, wherein said different species of bioconjugate molecule are hybridized to an ordered array of oligonucleotides immobilized to said support.

19. The method of claim 17, wherein said target cell is a mammalian cell, a reptilian cell, an avian cell, a fish cell, a fungal cell, a plant cell, a yeast cell, a bacterial cell, or a viral particle.

20. The method of claim 17, wherein said surface ligand is an antigenic surface protein, a receptor, a transmembranous enzyme, that is naturally present on the surface of normal target cells.

21. The method of claim 17, wherein the presence of said surface ligand is associated with a disease state.

22. The method of claim 17, wherein the ligand binding molecule is an immunoglobulin, a hormone, an immunomodulator, a cytokine, a chemokine, a pharmacological agent or a substrate or inhibitor of a transmembranous enzyme.

23. The method of claim 17, wherein said solid support is glass, paper, optical fiber, or plastic.

24. The method of claim 17, wherein said solid support is an optical waveguide, and wherein said detection of immobilization of target cells to said surface is preformed by measuring a detectable label using a fiber optic waveguide detector.

25. The method of claim 17, wherein a molecule of a species of one of said bioconjugate molecules is formed by a method that comprises the steps of:
(A) contacting an oligonucleotide having an amino group with a heterofunctional linker, wherein said linker has a first group reactive with said amino group and a second group reactive with a thiol group, said contacting being under conditions sufficient to permit said first group of said heterofunctional linker to become bonded to said amino group of said oligonucleotide, thereby forming an oligonucleotide-heterofunctional linker conjugate; and
(B) contacting said oligonucleotide-heterofunctional linker conjugate (A) with a protein having a thiol group reactive with said second group of said heterofunctional linker; said contacting being under conditions sufficient to permit said thiol group of said protein to become bonded to said second group of said heterofunctional linker of said oligonucleotide-heterofunctional linker conjugate, to thereby form a molecule of a species of said bioconjugate molecules.

26. The method of claim 25, wherein said first group of said heterofunctional linker is an NHS group, and said second group of said heterofunctional linker is a maleimide group.

27. The method of claim 25, wherein said heterofunctional linker is selected from the group consisting of Sulfo-SMCC; Sulfo-EMCS; Sulfo-GMBS; Sulfo-KMUS; Sulfo-MBS; Sulfo-SIAB; Sulfo-SMPB; Sulfo-LC-SMPT; SVSB; SIACX; SIA, SIAXX; and NPIA.

28. The method of claim 17, wherein said method further includes the step of determining whether any immobilized cells possess a desired internal molecule.

29. The method of claim 28, wherein said presence or expression of said internal component is characteristic of an apoptotic state or a disease state.

30. A method for identifying a ligand-binding molecule that binds to a surface ligand of a cell, said method comprising the steps:
(A) incubating a population of cells that possess said surface ligand in the presence of:
(1) a candidate ligand-binding molecule suspected of being capable of binding to said surface ligand of said cell;
(2) a bioconjugate molecule, said bioconjugate molecule comprising a ligand-binding molecule portion conjugated to an oligonucleotide molecule portion; and
(3) a support having immobilized thereto an oligonucleotide molecule;
wherein said bioconjugate oligonucleotide and said support-immobilized oligonucleotide are capable of hybridizing to one another, and wherein said incubation is conducted under conditions sufficient to permit:
(a) said bioconjugate oligonucleotide and said support-immobilized oligonucleotide to hybridize to one another; and
(b) said ligand-binding molecule portion of said bioconjugate to bind to said surface ligand of said cell; and
(B) determining whether the presence of said candidate ligand-binding molecule affects the extent of immobilization of said cells to said solid support.

31. A method for screening for a desired molecule comprising:
(A) incubating a candidate desired molecule in the presence of a microarray of cells immobilized to a solid support, wherein said microarray is formed by incubating a population of cells that possess a surface ligand in the presence of:
(1) a ligand-binding molecule capable of binding to said surface ligand of said cell;
(2) a bioconjugate molecule comprising a ligand-binding molecule portion conjugated to an oligonucleotide molecule portion; and
(3) a support having immobilized thereto an oligonucleotide molecule,
wherein said support has immobilized thereon an oligonucleotide molecule that hybridizes to the oligonucleotide portion of said bioconjugate and said ligand-binding molecule portion of said bioconjugate binds to said surface ligand of said cell so as to immobilize said cells to said solid support; and
(B) determining whether the presence of said candidate desired molecule affects the extent of immobilization of said cells to said solid support.
